SEQUENCE LISTING (7 sequences)

SEQ ID NO: 1 - DNA, 20 bp, Artificial Sequence
  Antisense oligonucleotide
gtctccgctccatcctgccc

SEQ ID NO: 2 - DNA, 25 bp, Artificial Sequence
  Antisense oligonucleotide
acgcagctcagtaacagtccgccta

SEQ ID NO: 3 - DNA, 19 bp, Artificial Sequence
  Antisense oligonucleotide
gggatcgttcagagtctag

SEQ ID NO: 4 - DNA, 26 bp, Artificial Sequence
  Antisense oligonucleotide
catcatggtcgtggtttgggcaaagc

SEQ ID NO: 5 - DNA, 22 bp, Artificial Sequence
  Antisense oligonucleotide
ccttagcggatgttgaccctgg

SEQ ID NO: 6 - DNA, 24 bp, Artificial Sequence
  Antisense oligonucleotide
acttgaacttcactcggccagcgg

SEQ ID NO: 7 - DNA, 25 bp, Artificial Sequence
  Antisense oligonucleotide
tcccaaggaaatgcagcttctgctt

(19) United States
(12) Patent Application Publication
(10) Pub. No.: US 2010/0310473 A1
(43) Pub. Date: Dec. 9, 2010

(54) Targeting Brain Cells Via Ophthalmic Delivery

(75) Inventors: Philip K. Liu, Winchester, MA (US); Christina H. Liu, Winchester, MA (US)

Correspondence Address: QUARLES & BRADY LLP, 411 E. WISCONSIN AVENUE, SUITE 2040, MILWAUKEE, WI, 53202-4497, US

(73) Assignee: THE GENERAL HOSPITAL CORPORATION, Boston, MA (US)

(21) Appl. No.: 12/671,283

(22) PCT Filed: Jul. 30, 2008
(86) PCT No.: PCT/US2008/071581

§ 371 Date: Jun. 30, 2010

Related U.S. Application Data
(60) Provisional application No. 60/962,499, filed on Jul. 30, 2007.

(51) Int. Cl. A61K 49/18; A61K 31/7088; A61K 31/7105; A61P 25/28

(52) U.S. Cl. 424/9.323; 514/44 R; 424/9.1; 424/9.3; 424/9.32; 977/773; 977/906; 977/916; 977/927; 977/930

(57) ABSTRACT

It is disclosed here that nucleic acid-based agents can be delivered to the brain of a human or non-human animal having a leakage in the blood brain barrier by administering the agents through the eye. Brain tissues and cells can be imaged in vivo (e.g., by magnetic resonance imaging) by linking a contrast agent to a targeting nucleic acid that can hybridize to a target nucleic acid located at the brain site to be imaged and administering the contrast agentltargeting nucleic acid conjugate through the eye. Similarly, a nucleic acid based drug (e.g., as an antisense nucleic acid or a therapeutic agent linked to a targeting nucleic acid that can hybridize to a target nucleic located at a disease site in the brain) can be administered through the eye to treat a brain disease.

### CROSS-REFERENCE TO RELATED APPLICATION

This PCT application claims priority to U.S. Provisional Application No. 60/962,499 filed Jul. 30, 2007, which is incorporated herein by reference.

### STATEMENT REGARDING FEDERALLY SPONSORED RESEARCH OR DEVELOPMENT

This invention was made with United States government support awarded by the following agency: National Institutes of Health Grant Nos. RO1NS045845, R21NS057556, R21NS024235, P41RR14075, 5T32CA009502, and P01AT002048. The United States government has certain rights in this invention.

### BACKGROUND OF THE INVENTION

Changes in gene expression and genetic mutations have been linked to many diseases and physiological conditions. As a result, various nucleic acid molecules have been employed as diagnostic and therapeutic agents. Nucleic acids can be used as probes to detect gene expression and mutation, as expression vectors to express exogenous genes in target cells, as antisense therapeutic agents to inhibit gene expression, and as a vehicle to deliver other molecules such as diagnostic or therapeutic molecules linked thereto to target cells. For example, WO 2006/023888 (incorporated herein by reference in its entirety) described linking a medical imaging contrast agent to a nucleic acid probe for imaging gene expression in various tissues, e.g., by magnetic resonance imaging, including the brain of living subjects.

The blood-brain barrier (BBB) is a membranic structure that acts primarily to protect the brain from chemicals in the blood, while still allowing essential metabolic function. It is composed of endothelial cells, which are packed very tightly in brain capillaries. In its neuroprotective role, the blood-brain barrier functions to hinder the delivery of many diagnostic and therapeutic agents to the brain, including nucleic acids.

In the injured or diseased brain, nucleic acid-based agents can play an important role in diagnosis or treatment. However, to be effective, such agents must cross the BBB and enter the brain. Brain injury caused by cardiac arrest, stroke, or diseases such as meningitis, multiple sclerosis, cancer, or Alzheimer's Disease, can result in leakage of the BBB. When the BBB is compromised by injury or disease, alternative methods to deliver nucleic acids and other diagnostic or therapeutic agents may become available. The inventors have developed a non-invasive method of delivering nucleic acid-based agents into a human or non-human animal having a leakage in the BBB.

### BRIEF SUMMARY OF THE INVENTION

It is disclosed here that nucleic acid-based agents can be delivered to the brain of a human or non-human animal having a leakage in the blood brain barrier (BBB) by administering the agents through the eye.

In one aspect, the present invention relates to a method for delivering a targeting nucleic acid to the brain tissue of a living human or non-human animal having a leakage in the blood brain barrier wherein the targeting nucleic acid is designed for hybridizing to a target nucleic acid (e.g., a target gene genomic sequence and/or its mRNA transcript) in the brain. The method includes the step of administering an active agent that comprises the targeting nucleic acid to an eye of the human or non-human animal wherein the agent travels to the brain tissue of the human and non-human animal and hybridizes to the target nucleic acid if present in the brain.

In another aspect, the present invention relates to a method for imaging a targeting nucleic acid or a target nucleic acid in the brain of a living human or non-human animal having a leakage in the blood brain barrier. The method includes the steps of providing an active agent that comprises a contrast agent linked to the targeting nucleic acid designed to hybridize to the target nucleic acid (e.g., the target gene genomic sequence and/or its mRNA transcript), administering the active agent to an eye of the human or non-human animal in an amount sufficient to provide a detectable image, allowing sufficient time to pass to allow unhybridized active agent to leave the brain, and imaging the brain wherein a detectable image of the contrast agent in the brain indicates the presence of cells containing the target nucleic acid in the brain. Thus in one embodiment, the method can be used to identify the presence of cell types producing a specific type of mRNA and identifying and studying developmental processes wherein specific types of mRNA are produced.

In another aspect, the present invention relates to a method for decreasing the expression of a target gene in the brain of a living human or non-human animal having a leakage in the blood brain barrier. The method includes the step of administering an active agent comprising a targeting nucleic acid to an eye of the human or non-human animal in an amount sufficient to decrease the expression of the target gene wherein the targeting nucleic acid can hybridize to a target nucleic acid corresponding to the target gene (e.g., the target gene genomic sequence and/or its mRNA transcript) and the hybridization between the targeting nucleic acid and the target nucleic acid leads to decreased expression or gene action of the target gene.

In another aspect, the present invention relates to a method for treating a disease or disorder in the brain of a living human or non-human animal having a leakage in the blood brain barrier. The method includes the step of administering an active agent to an eye of the human or non-human animal in an amount sufficient to treat the disease or disorder wherein the active agent comprises a targeting nucleic acid and a therapeutic agent linked together and the targeting nucleic acid can hybridize to a target nucleic acid (e.g., the target gene genomic sequence and/or its mRNA transcript) located at the site of the disease or disorder. In another aspect, the present invention relates to a method for delivering a nucleic acid comprising a nucleotide sequence encoding a polypeptide to be expressed in the brain (e.g., in a brain cell) to the brain of a human or non-human animal having a leakage in the blood brain barrier. The method includes the step of administering an active agent comprising the nucleic acid to an eye of the human or non-human animal in an amount sufficient to express the polypeptide. In one embodiment, the nucleic acid further comprises a promoter operably linked to the nucleotide sequence. The expression of the polypeptide may be for the purpose of treating a disease or disorder in the brain.

### BRIEF DESCRIPTION OF THE SEVERAL VIEWS OF THE DRAWINGS

FIG. 1 shows detection of scar formation in live mice using both immunochemistry techniques (Panel A) and SPION-actin by MRI (SPION stands for superparamagnetic iron oxide nanoparticles) wherein the contrast agent is delivered via the orbital route (Panels B and C). A group of six C57black6 mice were wounded by puncture in the left cortex. Three days later, one half of the group (n=3) were sacrificed for immunohistochemical assessment of the wounds. The other half were kept alive for at least eight weeks for wound detection using SPION-actin by MRI. Panel A shows the results of the immunohistochemical assessment. Postmortem histology shows gliosis in the tissue surrounding an intracranial puncture site (left side) but not in the contralateral hemisphere (right side). Antibodies against glial fibrillary acidic protein (GFAP) (Alkaline phosphotase-labeled) were used to detect glia and astrocytes. Panel B shows T2-weighted (T2w) magnetic resonance imaging (MRI) of the animal after needle induced brain injury (8 weeks). MRI slice thickness is 1 mm. The arrow points to the area of injury. Panel C shows R2* maps of the same animal (MRI slice thickness is 0.5 mm) using MR contrast agent targeting GFAP expressing cells (T2 susceptibility agent with sODN-gfap, sODN stands for phosphorothioate-modified oligodeoxynucleotide), which has a sequence complementary (antisense) to glial fibrillary acidic protein (sODN-gfap, 5′-gtctccgctccatcctgccc-3′, SEQ ID NO:1, 6 KD) mRNA of the mouse (S. A. Lewis et al., Proc Natl Acad Sci USA 81, 2743, 1984). The arrow points to cells that express gfap mRNA around the injured site (dark gray area). We delivered SPION-gfap-FITC (4 mg Fe per kg in 0.1 ml) by an eye dropper (20 μl every 15 min under ketamine [100 mg/kg] and xylazine [10 mg/kg] anesthesia). Imaging was done one day post delivery.

FIG. 2 shows detection of small brain scar in live mice after global cerebral ischemia by bilateral carotid artery occlusion (BCAO) using SPION-gfap by MRI and evolution of brain damage after BCAO using DWI/ADC and T2W MRI. We induced BBB leakage by BCAO (60 minutes) in C57Black6 mice. DWI/ADC (diffusion weighted image/apparent diffusion coefficient) MRI was obtained at one day of reperfusion to show where brain injury would be present (panel A-i). We applied SPION-gfap as eye drops at eight weeks after BCAO, and R2* maps were obtained the next day (panel A-ii). T2 MRI was obtained at the same time for comparison (panel B). Respective images and maps of sham-operated mice are in panels C (DWI one day after sham operation) and D (R2* maps eight weeks after sham operation). Evidence of BBB leakage after BCAO is shown by pre- and post-gadolinium-EDTA (0.1 mmol/kg, IV) and MRI in vivo (panels A-iii and A-iv, respectively). DWI hyperintensity, elevated R2* maps, and BBB leakage are indicated by arrows.

FIG. 3 shows cell typing at the scar by SPION-sODN and transcription MRI. We applied SPION-actin and SPION-gfap using eye droppers to five animals previously treated with BCAO (after eight and nine weeks, respectively, at 10 mg Fe per kg). We acquired R2* maps the next day after the application (one of 5 animals is shown). A magnified view is present to the right of each image. (A) T2 weighted MR image reveals injured sites at the dentate gyms (arrows and circles). (B) A lack of SPION-actin retention at the location ventral to the injured site eight weeks after injury induction. (C) A presence of SPION-gfap retention at the injured site (circle) nine weeks after injury induction. (D) A lack of SPION-mmp9 retention at the location ventral to the injured site eleven weeks after injury induction. (E) Immunohistochemistry of postmortem tissue of the same animal imaged in panels B and C for actin and GFAP expressing cells in the hippocampus. (F) This panel is an anatomical drawing of the brain cross-section imaged in the left side of panels A-D. The SPION-actin retention profile shown in FIG. 3 indicates preferential retention in regions near the scar or the region undergoing repair after BCAO. Actin is expressed during skin scarring if no pressure is applied (Costa A M et al. Am J Pathol 155, 1671-1679, 1999) and actin is one of many genes expressed in CNS microvascular pericytes (Dore-Duffy P et al. J Cereb Blood Flow Metab 26, 613-624, 2006). Angiogenesis during repair of injury involves the expression of MMP-9 and actin. Our in vivo MRI agrees with these reports for cell typing at the injury site.

FIG. 4 shows delivery of SPION-actin to neurons via OTR delivery after cortical spreading depression in C57black6 mice (Gursoy-Ozdemir Y et al. J Clin Invest 113, 1447-1455, 2004). This model induces BBB leakage in the brain without causing scar in the brain. SPION-actin-FITC (10 mg/kg, eye droppers, FITC is attached to targeting nucleic acids for histology tracking) was delivered five days after cortical spreading depression; R2* maps were obtained the next day (panel A). The images in each row go from posterior on the left side to anterior on the right side. The top row contains BBB-1 images, the second row contains BBB-2 images, the third row contains Sham-1 images, and the bottom row contains Sham-2 images. Subtraction R2* maps show global retention of SPION (Panel B), with representative elevations of the R2* maps calculated as (Post OTR MRI-pre OTR MRI of BBB-1 in Panel A)×100%. Statistical analysis of SPION-retention is shown in panel C, which compares three regions of interest in both hemispheres (hippocampus, striatum, and SSC) in BBB and sham mice.

FIG. 5 shows histological photographs showing that animals with BBB leakage (by cortical spreading depression) have higher FITC (targeting nucleic acids) than the animal without BBB leakage (sham-operation). Histology of CA1 neuronal formation of the hippocampus was taken after OTR of SPION-actin-FITC. Mouse cells were stained using Texas Red-murine monoclonal IgG to provide enhanced contrast for FITC in SPION-actin (lighter shade). Panel A shows probe uptake in BBB-1. Panel B shows probe uptake in Sham-1.

FIG. 6A shows the location of fosB mRNA from which the sequence in sODN-delta fosB (ΔfosB) is designed so that it is complementary to its mRNA. FIG. 6B shows the location of ΔFosB mRNA from which we designed the complementary sequence of sODN-ΔfosB. FIG. 6C shows the specificity of SPION-fosb and SPION-ΔfosB. Polymerase chain reaction is used to amplify a partial FosB (146 basepairs) and a partial ΔFosB (123 basepairs) as defined in panels 6A & 6B from a fragment of Fos B (584 basepairs) and ΔFosB cDNA (434 basepairs) (after BamH1 and PstI cut to FosB or ΔfosB mRNA) in the presence of the unique upstream sODN. After PCR amplification (94° C.; 30 sec; 37° C.; 45 sec; 50° C.; 30 sec, ×30), we observed sODN-fosB generated a 146 basepair-fragment while sODN-ΔfosB generated a 123 basepair-fragment. When the BamH1-PstI cut DNA fragments were hybridized to the cDNA with FITC sODN-fosB or with FITCsODN-ΔfosB, we observed specific hybridization of these sODN to its own fragment (FIG. 6D). Panel E shows SPION retention in the brain representing baseline (no SPION infusion), the endogenous level of c-fos mRNA, fosB mRNA and ΔfosB mRNA. Regional R2* (SPION uptake) was measured seven hours post ICV infusion.

FIG. 7 shows SPION-fosB detected an elevation of fosB mRNA in the brain in living mice (Panels B & C) after acute amphetamine exposure (4 mg/kg. i.p.) in a group of mice that had no prior experience with drug of abuse (acute challenge in FIG. 7A-top). Panel A outlines the amphetamine treatments in studies involving acute amphetamine challenge (top) and in studies involving chronic amphetamine exposure (bottom). Panel B shows R2* maps of SPION-fosB in live mouse brains after acute amphetamine challenge (bottom row) and no amphetamine challenge (vehicle; top row). Panel C shows subtraction R2* maps of the SPION-fosB images from panel B. Shading indicates percent increase relative to vehicle.

FIG. 8 shows additional data from the acute amphetamine challenge studies shown in FIG. 7. Bar graph in FIG. 8A shows a quantitative comparison in SPION-fosB between vehicle mouse brains and mouse brains after acute amphetamine challenge in various regions of interest. The results show that fosB mRNA was specifically increased after acute amphetamine challenge in the pleasure pathway of the brain. Panel B shows elevated fosB mRNA in the R2* maps of acute amphetamine challenged mice (bottom) as compared to vehicle mice (top). Panel C shows a parallel increase in FosB-FITC fluorescein probe uptake in the cytoplasm of the accumbens nucleus shell (part of the pleasure pathway) in acute amphetamine challenged mice as compared to vehicle mice (top).

FIG. 9 shows fosB mRNA was not elevated further in animal brain after repeated exposures to amphetamine (chronic exposure, see protocol outlined in FIG. 7A-bottom). Panel A shows R2* SPION-fosB maps of live mouse brains from mice subject to amphetamine challenge after previous chronic amphetamine exposure (bottom) as compared to naïve (no previous amphetamine exposure) mice subject to amphetamine challenge (top). The bar graph in Panel B shows a quantitative comparison in fosB in various regions of interest between mouse brains subject to amphetamine challenge after chronic amphetamine exposure (sensitized) and mouse brains subject to amphetamine challenge without previous exposure (naive).

FIG. 10 shows SPION-ΔfosB was not elevated in the pleasure pathway of the brain after acute amphetamine exposure (4 mg/kg, i.p.). Panel A shows R2* SPION-ΔfosB maps of live mouse brains from naïve mice subject to acute amphetamine challenge (bottom) and vehicle control mice (top). The bar graph in Panel B shows a quantitative comparison in SPION-ΔfosB between mouse brains subject to acute amphetamine challenge and vehicle mouse brains in various regions of interest.

FIG. 11 shows that amphetamine exposure to a mouse previously and repeatedly being exposed to the drug (sensitized, or chronic user) increases ΔFos B mRNA detected using SPION-ΔfosB. Panel A shows R2* SPION-ΔfosB maps of live mouse brains from naïve mice subject to amphetamine challenge (top) and from sensitized (mice subject to chronic amphetamine exposure) mice subject to amphetamine challenge (bottom). The bar graph in Panel B shows a quantitative comparison of R2* SPION-ΔfosB mRNA in various regions of interest between mouse brains subject to amphetamine challenge without previous exposure (naïve) and mouse brains subject to amphetamine challenge after chronic amphetamine exposure (sensitized).

FIG. 12 shows that the elevation in Δfos gene expression is confirmed by increase in translation of Δfos B antigen in sensitized animals. Panel A shows the results of Δfos antigen detection in the NAc, core, and shell of the nucleus accumbens in sensitized (left side, n=6) and naïve (right side, n=6) challenged mice. Panel B shows the results of Δfos antigen detection in the cigulate and medial prefrontal cortices in sensitized (left side, n=6) and naïve (right side, n=6) challenged mice. The sensitized animals have been shown by others to have behavior abnormal syndromes similar to those of humans.

FIG. 13 shows a combination therapy for weight loss using amphetamine and acupuncture. Panel A shows body weigh drop in mice (n=4) was observed with repeat exposures to amphetamine (4 mg/kg daily every other days for 14 days, i.p.) and repeat exposures to amphetamine plus acupuncture treatment (*p<0.04, compared to saline). Panel B shows that although body weight loss associated with repeat exposures to amphetamine was not reversed by acupuncture, ΔfosB mRNA expression (detected by SPION-ΔfosB) was. The bar graph in Panel B shows a quantitative comparison of R2* SPION-ΔfosB mRNA between mouse brains challenged after chronic amphetamine exposure (sensitized) and mouse brains challenged after chronic amphetamine exposure followed by acupuncture treatment (sensitized-acupuncture) in various regions of interest.

FIG. 14 shows in vivo detection of brain cells expressing nestin mRNA using SPION-nestin delivered to neurons via eyedrop delivery after inducing global cerebral ischemia (GCI) in C57black6 mice by bilateral occlusion of the common carotid arteries (BCAO) for 60 minutes. Panel A shows brain edema detected by DWI/MRI at two days after GCI. Panel B shows R2* SPION-nestin expression profiles in the subgranular layer and subventricular zone four week after GCI recorded one day following probe delivery via eyedrops (2 mg Fe/Kg).

FIG. 15 is a subventricular zone (SVZ) ×20 histological image from mouse brain showing nestin antigen-stained stem cells (sharp bright spots) and gfap stained cells and nuclei (diffuse darker grey spots). SPION-nestin was delivered to neurons via intraperitoneal injection after inducing GCI for 30 minutes.

FIG. 16 is a subventricular zone (SVZ) ×20 histological image from brains of sham-operated mice showing gfap stained cells and nuclei (diffuse dark grey spots). No nestin staining (sharp bright spots) was observed.

### DETAILED DESCRIPTION OF THE INVENTION

The present invention is based, in part, on the inventors' discovery that agents containing nucleic acid molecules can be delivered to brain cells through the eye in animals with blood brain barrier (BBB) leakage. Using superparamagnetic iron oxide nanoparticles (SPION) conjugated nucleic acid probes that target the gene transcript of glial fibrillary acidic protein (gfap), actin, matrix metalloproteinase-9 (mmp-9), the inventors have shown in the examples below that these agents can be delivered to target brain cells via eye drops in mice with BBB leakage and MRI images can be obtained for brain cells that express gfap, actin, or mmp-9. In an additional proof of principle example, the inventors have shown that SPION probes targeting the gene transcript of nestin can be noninvasively delivered to target brain cells in mice with BBB leakage and that MRI images can be obtained for brain cells that express nestin.

GFAP protein is a major component of brain scar tissue (e.g., at injured brain sites) and of glioma and it is expressed in glia and astrocytes (the major cell types in glioma) and in slowly proliferating type B cells, but not in neurons; actin is expressed in all brain cell types (e.g., neurons and microvascular cells of multipotential stem cells), except glia and astrocytes, and is a marker for angiogenesis in the brain (e.g., neural repair of damage that involves angiogenesis); mmp-9 is expressed in brain cells and is a marker for stroke, brain damage by heart attack, and angiogenesis during stroke repair and tumor metastasis; and nestin is expressed in stem cells such as slowly proliferating type B cells and actively proliferating type C cells.

As BBB leakage occurs in most brain diseases such as infection, neurological diseases, and brain tumors, the disclosure here enables new tools for delivering nucleic acid based agents to the brain for the purpose of diagnosing, treating, or preventing these diseases. The ophthalmic route of delivery is noninvasive and can sometimes be administered by a patient at home. For example, a patient may administer SPION conjugated nucleic acid via eye drops at home the day before going to the hospital for MRI detection of certain brain damages. The eye delivery method of the present invention can also be applied to a human or non-human animal who does not already have a leakage in the BBB. In this case, a transient BBB leakage can be induced by the various known methods. For example, osmotic shock (manitol 1.6 M, intravenous infusion, B T Hawkins and R D Egleton, J Neuroscience Methods 151, 262-267, 2006) can be used to induce temporary BBB leakage for delivery of active agents via eye drops. Other examples include the use of toxin or microwave.

In general, the present invention provides a method of delivering an agent that comprises a nucleic acid or a contrast agent to the brain of a human or non-human animal having a BBB leakage via the ophthalmic route. Examples of non-human animals include non-human primates, monkeys, rats, mice, pigs, horses, sheep, goats, cattle, cats, and dogs. For the purpose of the present invention, an agent that comprises a nucleic acid such as a targeting nucleic acid or an in vivo medical imaging contrast agent is referred to as an active agent. The terms “ophthalmic route/delivery,” “ocular route/delivery,” and “orbital route/delivery” are used interchangeably to refer to administering an agent through the eye, preferably as eye drops.

One skilled in the art is familiar with the brain diseases that cause a leakage in the BBB. BBB leakage of the brain can be readily detected by any of the known methods in the art. For example, gadolinium or DWI/ADC MRI is routinely administered in clinical settings (e.g., Gd-DTPA (diethylenetriamine pentaacetic acid gadolinium)) to detect BBB leakage.

An active agent of the present invention can be delivered to target brain cells, which include normal and abnormal brain cells such as brain tumor cells.

Many brain diseases can be imaged by delivering a contrast agent to target cells in the brain through the eye. In this regard, the contrast agent is linked to a targeting nucleic acid which can hybridize to a target nucleic acid in the target cells so that the contrast agent can be retained by the target cells. For example, a targeting nucleic acid can be designed to hybridize to an mRNA expressed in the target cells but not in other cells or to an mRNA that is expressed at a higher level in the target cells than in other cells. Examples of such diseases include glioma, brain necrosis of scar tissue (stroke, heart disease, or head traumatic injury), Alzheimer's disease, multiple sclerosis (MS), viral infection such as HIV/AIDS and viral encephalitis, Huntington's disease, and Parkinson's disease. One skilled in the art is familiar with the genes in these diseases that can be targeted by the targeting nucleic acid for imaging. For example, for glioma and brain scar tissue, gfap mRNA can be targeted by the targeting nucleic acid; for Alzheimer's disease, gfap mRNA and/or beta amyloid precursor protein mRNA can be targeted by the targeting nucleic acid; for MS, gfap mRNA can be targeted by the targeting nucleic acid; and for viral infection, a viral gene transcript (mRNA) can be targeted by the targeting nucleic acid (e.g., for HIV/AIDS, an HIV gene transcript can be targeted by the targeting nucleic acid). In addition, mmp-9 mRNA can be targeted by the targeting nucleic acid to image stroke, brain damage by heart attack, angiogenesis during stroke repair, and angiogenesis during brain tumor metastasis.

The method described above can be used to diagnose a brain disease if a disease-specific mRNA is targeted. If a marker for various brain diseases such as gfap mRNA is targeted, the method can be used to assist diagnosis of a particular brain disease in connection with other known diagnostic methods for the disease. c-fos mRNA is another marker that can be targeted by the targeting nucleic acid for imaging several brain diseases or normal conditions such as brain injury induced by cardiac arrest or cerebral ischemia and the effect of amphetamine (Liu C H et al., Mol Imaging, 6, 156-170, 2007; and Liu C H et al. FASEB, in press, E-publication: May 30, 2007; and Liu C H et al., J Neurosci, 27, 713-722, 2007, all of which are herein incorporated by reference in their entirety). Normal conditions include expression of hippocampal c-fos mRNA (neuronal activation) during learning process. Similar elevation in c-fos expression also occurs when the pleasure pathway is stimulated such as by amphetamine or cocaine exposure, eating, or having sex.

Many physiological and/or pathological processes can also be imaged. For example, neurofilament-1 mRNA can be targeted by the targeting nucleic acid for imaging neurogenesis. Stem cell-specific growth factor mRNA such as epidermal growth factor (EGF) mRNA can be targeted by the targeting nucleic acid for imaging stem cell activity. Neurogenesis and stem cell activity can be imaged by using the targeting nucleic acid to target nestin mRNA. Actin mRNA can be targeted by the targeting nucleic acid for imaging angiogenesis in the brain.

Furthermore, many brain diseases can be treated by delivering a therapeutic agent (e.g., those known to be able to treat the diseases) to target cells in the brain through the eye. In this regard, the therapeutic agent is linked to a targeting nucleic acid which can hybridize to a target nucleic acid in the target cells so that the therapeutic agent can be delivered to the target cells. For example, a targeting nucleic acid can be designed to hybridize to an mRNA expressed in the target cells but not other cells or to an mRNA that is expressed at a higher level in the target cells than in other cells. Examples of such diseases include glioma, brain injury (brain scar tissue), Alzheimer's disease, multiple sclerosis (MS), viral infection such as HIV/AIDS and viral encephalitis, Huntington's disease, and Parkinson's disease. One skill in the art is familiar with the genes in these diseases that can serve as deliver targets and the therapeutic agents that can be delivered to treat the diseases. For example, for glioma, gfap mRNA can be targeted by the targeting nucleic acid and the therapeutic agent linked to the targeting nucleic acid can be a cytotoxic agent or chemotherapeutic agent; and for viral infection, a viral gene genomic sequence or its mRNA can be targeted by the targeting nucleic acid and the therapeutic agent linked to the targeting nucleic acid can be a cytotoxic agent or antiviral agent (e.g., for HIV/AIDS, an HIV gene genomic sequence or its mRNA can be targeted by the targeting nucleic acid and the therapeutic agent linked to the targeting nucleic acid can be a cytotoxic agent or antiviral agent).

Certain brain diseases can be treated by inhibiting the expression of a particular gene in target cells. In this regard, an antisense nucleic acid can be delivered to the target cells in the brain through the eye to inhibit the expression of the target gene. As an example, polyADP-ribose polymerase expression can be inhibited for treating stroke. As another example, a viral gene genomic sequence or its mRNA can be targeted for treating viral infection (e.g., HIV/AIDS).

Certain other brain diseases can be treated by expression a polypeptide such as a protein in the brain tissue or brain cells. In this regard, a nucleic acid comprising a nucleotide sequence encoding the polypeptide to be expressed is administered to an eye of the human or non-human animal. In one embodiment, the nucleic acid further comprises a promoter operably linked to the nucleotide sequence. For example, various growth factors can be expressed in the brain to treat neurodegenerative diseases.

Active Agents:

An active agent that can be delivered to the brain via the ophthalmic route as provided here contains a targeting nucleic acid (e.g., DNA or RNA or derivatives of naturally occurring DNA and RNA), a targeting nucleic acid linked to a contrast agent, a targeting nucleic acid linked to a therapeutic agent, or a targeting nucleic acid, a contrast agent, and a therapeutic agent linked together. The targeting nucleic acid is designed to hybridize or bind to a target nucleic acid (e.g., a target gene and/or its gene transcripts) in the brain. Preferably, the targeting nucleic acid hybridizes or binds “specifically” to the target nucleic acid, i.e., it hybridizes or binds preferentially to the target nucleic acid and does not substantially bind to other molecules or compounds in the brain. Optionally, an active agent according to the present invention can further contain a localization molecule, which can be linked to the targeting nucleic acid, the contrast agent, or the therapeutic agent.

Various components of the active agent can be linked together either covalently (e.g., directly through a chemical bond or indirectly through a linker and chemical bonds) or noncovalently (e.g., through ionic, van der Waals, electrostatic, or hydrogen bonds). One skilled in the art is familiar with the methods that can be used to link these components. Some examples are provided here and more can be found in US patent application publication US 2003/0049203, which is herein incorporated by reference in its entirety.

The target nucleic acid may inhibit the transcription of a target gene, serve as a probe for the expression of a target gene, assist in localizing the active agent in a target region or cell in the brain, promote retention of the active agent by a target cell, or any combination thereof. In some embodiments, the targeting nucleic acid has 10-100 nucleotides, 12-100 nucleotides, 12-60 nucleotides, 14-50 nucleotides, 14-40 nucleotides, 14-35 nucleotides, 14-30 nucleotides, or 17-35 nucleotides.

The term “nucleic acid” includes single, double, and triple stranded molecules and nucleic acid molecules that are chemically or enzymatically modified. Various types of modifications such as those for providing nuclease resistance are well known in the art. Some of these modifications are described in US patent application publication US 2003/0049203, which is herein incorporated by reference in its entirety.

The targeting nucleic acid can be an antisense nucleic acid (e.g., an oligonucleotide or 12-35 or 14-30 nucleotides), an RNA interference (RNAi) molecule such as an siRNA (small interfering RNA) molecule, or an shRNA (short hairpin RNA) molecule for inhibiting the expression of specific genes in targeted brain regions or cells. When delivered to the target region (e.g., viral infection site) or brain cells, the targeting nucleic acid can inhibit the expression of target genes, which may lead to disease treatment or prevention. When the active agent also comprises a contrast agent linked to the targeting nucleic acid, the delivery of the targeting nucleic acid can be tracked by medical imaging. When the active agent also comprises a therapeutic agent linked to the targeting nucleic acid which by itself has therapeutic activity through inhibiting gene expression, the therapeutic effects of the targeting nucleic acid molecule and the therapeutic agent may complement each other to achieve better therapeutic results.

A targeting nucleic acid at least part of which is complementary to a part of an RNA transcribed from a target gene such as an mRNA in a target brain cell can be used to detect the expression of the gene, localizing the active agent in the target brain region or cells, or promote the retention of the active agent by the target brain region or cells. When the active agent comprises a contrast agent in addition to the targeting nucleic acid, the expression of the gene and the target brain region or cells can be imaged. When the active agent comprises a therapeutic agent such as a cytotoxic or chemotherapeutic agent in addition to the targeting nucleic acid, certain therapeutic effect can be achieved, e.g., by killing the targeted cell. By complementary nucleic acid, we mean sequences which have sufficient complementarities to be able to hybridize to each other under highly stringent or mildly stringent hybridization conditions. If the nucleotide sequence of a nucleic acid molecule is sufficiently long, one or more mismatches can be tolerated. One skilled in the art can readily determine the degree of mismatching which may be tolerated based upon the melting point, and therefore the thermal stability, of the resulting duplex. Stringent hybridization conditions are defined as hybridizing at 68° C. in 5×SSC/5×Denhardt's solution/1.0% SDS, and washing in 0.2×SSC/0.1% SDS+/−100 μg/ml denatured salmon sperm DNA at room temperature, and moderately stringent hybridization conditions are defined as washing in the same buffer at 42° C. Additional guidance regarding such conditions is readily available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

Depending on the specific applications, a targeting nucleic acid may be designed to hybridize (e.g., preferentially hybridize) to any gene or its corresponding mRNA that is known to be specifically expressed or differentially expressed (i.e., higher level of expression) in a group of brain cells to allow the cells to be imaged, a disease associated with the expression of the gene to be treated, or both.

Contrast agents that can be employed in an active agent according to the invention are those useful in various known in vivo medical imaging modalities such as X-ray imaging, ultrasound imaging, computed tomography (CT) imaging, diffuse optical tomography (DOT) imaging, magnetic resonance imaging (MRI), and nuclear medicine imaging such as positron emission tomography (PET) imaging and single photon emission computed tomography (SPECT) imaging. Many of these imaging modalities and their corresponding contrast agents are described in Min J J et al. (Gene therapy, 11:115-125, 2004), which is herein incorporated by reference in its entirety. Others are also known in the art. These contrast agents are detectable e.g., by emitting light, radioactive emissions, or chemical signals, by absorbing radiation (e.g., x-rays), or by otherwise changing a characteristic of targeted cells relative to other cells. Examples of contrast agents include chemiluminescent compounds, radioisotopes/radionuclides, fluorescent molecules, paramagnetic contrast agents, and metal chelates. Specific examples of contrast agents (radionuclides) for PET or SPECT include ¹³¹I, ¹²⁵I, ¹²³I, ^{99m}Tc, ¹⁸F, ⁶⁸Ga, ⁶⁷Ga, ⁷²As, ⁸⁹Zr, ⁶⁴Cu, ⁶²Cu, ¹¹¹In, ²⁰³Pb, ¹⁹⁸Hg, ¹¹C, ⁹⁷Ru, and ²⁰¹Tl. Specific examples of contrast agents for MRI include paramagnetic contrast agents such as gadolinium, cobalt, nickel, manganese, and iron. As used herein, “paramagnetic” means having positive magnetic susceptibility and lacking magnetic hysteresis (ferromagnetism).

Therapeutic agents that can be included in an active agent according to the present invention include those that can counter an abnormal condition in the brain (e.g., tumor or infection). Examples of such therapeutic agents include enzymes, enzyme inhibitors, receptor ligands, radioisotopes, antibiotics, and polypeptides. Any suitable therapeutic agents known in the art can be employed. In one embodiment, a cytotoxic agent or a chemotherapy agent is employed.

A localization molecule, when included in an active agent according to the present invention, assists the active agent in localizing to a particular target area in the brain, entering a target brain cell, and/or binding to a receptor such as a cell surface receptor on a target brain cell, all of which will assist the active agent to reach or enter the target area or cell more efficiently. One skilled in the art is familiar with the localization molecules that can be used (US patent application publication US 2003/0049023, which is herein incorporated by reference in its entirety). Examples include internalizing peptides, antibodies (including fragments and functional equivalents thereof) to a cell surface receptor on a brain cell, and ligands of a cell surface receptor on a brain cell. Preferably, the receptor mediates endocytosis. Brain cells are known to have surface receptors that can recognize specific sugar molecules and such sugar molecules can serve as localization molecules. For example, an active agent can be glycosylated with one or more mannose residues to yield an active agent having higher affinity binding to glioblastoma and gangliocytoma cells expressing mannose receptors.

An active agent according to the present invention may also be one that comprises a nucleic acid having a nucleotide sequence encoding a polypeptide to be expressed in the brain (e.g., in a brain cell). In one embodiment, the nucleic acid further comprises a promoter operably linked to the nucleotide sequence. Optionally, a contrast agent can be linked to the nucleic acid for monitoring the delivery of the active agent to the brain.

An active agent according to the present invention may also be one that contains a contrast agent described above linked to a non-nucleic acid molecule such as a polypeptide for targeting a site of disease or disorder in the brain (e.g., by binding to target molecule located at the disease/disorder site) and many of such agents are known in the art. For example, U.S. patent application publication 20060110323 (herein incorporated by reference in its entirety) describes using a radionuclide-labeled compound that comprises a C2 domain of a protein or an active variant thereof for imaging cell death. In general, any active agent that comprises a contrast agent known to be suitable for imaging a physiological or pathological condition in the brain can be delivered via the ophthalmic route. An active agent containing a contrast agent and a targeting molecule linked together in an amount sufficient to provide a detectable image can be administered and sufficient time is allowed to pass for the unbound active agent to leave the brain. The brain can then be imaged for the disease or disorder wherein a detectable image of the contrast agent in the brain indicates the presence of target molecule or the disease/disorder.

Delivery Method:

It is well within the capability of a skilled artisan to formulate an active agent of the present invention for delivery through the ophthalmic route. General guidance can be found in Remington's Pharm. Sci., 19th Ed., Mack Publ. Co., 1995, which is herein incorporated by reference in its entirety. For example, a composition comprising an active agent and a suitable pharmaceutically acceptable carrier can be administered through the eye. Additional agents such as nuclease inhibitors for stabilizing nucleic acid molecules may also be included in the composition. For delivering nucleic acid molecules such as DNA molecules, it is well known in the art that both viral based and non-viral based systems are available. Examples of viral based systems include retrovirus, lentivirus, adenovirus, and herpes simplex virus vectors. Examples of non-viral systems include naked DNA and liposomes. It is well known in the art that liposome is a efficient vehicle for introducing agents, especially agents that comprise a nucleic acid moiety, into cells. Various viral based and non-viral based systems are described in US patent application publication US 2002/0192688, which is herein incorporated by reference in its entirety. As another example, nucleic acids such as DNA molecules can be compacted (by polymers) to a size range of 10-30 nm for delivery (Liu G et al., J Biol Chem 278, 32578-86, 2003).

In a preferred embodiment, an active agent according to the present invention is provided in a liquid suspension so that it can be conveniently administered as eye drops. Other types of formulation such as an ointment containing an active agent may also be employed. Depending on the chemical property of a particular active agent such as whether it is water soluble and whether it is provided in a liposome or viral vector, the liquid suspension can be in the form of a solution, a colloid (particles of 1 to 100 nm in size dispersed in a continuous liquid phase), an emulsion (oil in water or water in oil with droplets over 100 nm in size), or a microemulsion (oil in water or water in oil with droplets of 100 nm or smaller in size). For example, for formulations containing a weakly water soluble active agent, microemulsions may be employed, e.g., by using a nonionic surfactant such as polysorbate 80 in an amount of 0.04-0.05% (w/v), to increase solubility.

Examples of suitable ophthalmic solutions are described below and other types of liquid suspensions can be made similarly.

An active agent that is delivered according to the method of the present invention can be provided in a buffered, isotonic ophthalmic solution in an amount that is pharmaceutically effective. Such a solution can be prepared by mixing the agent with pharmaceutically acceptable carriers, fillers, diluents, and the like. Preferably, the solution is sterile.

For example, an active agent can be added to, optionally, a base material solvent and then made into an aqueous solution or a suspension. Various additive agents may be optionally added to the ophthalmic solutions, such as buffer agents (e.g., phosphate buffer agents, borate buffer agents, citrate buffer agents, tartarate buffer agents, acetate buffer agents, amino acids, and the like), tonicity/isotonic agents (e.g., sorbitol, glucose, mannitol, other suitable saccharides, glycerin, sodium chloride and the like salts, and the like), antiseptics/preservatives (e.g., benzalkonium chloride, benzethonium chloride, parabens, benzyl alcohol, thimerosal, chlorobutanol, phenylethyl alcohol, edetate disodium, sorbic acid, potassium sorbate, polyquarternium-1, and the like), pH adjusting agents (e.g., hydrochloric acid, phosphoric acid, sodium hydroxide, and the like), thickeners (e.g., hydroxyethyl cellulose, hydroxypropyl cellulose, methyl cellulose, hydroxypropylmethyl cellulose, carboxymethyl cellulose, salts of any of the forgoing, and the like), solubilizing agents (e.g., ethanol, polyoxyethylene hydrogenated castor oil, Polysorbate 80, and the like), and the like. In addition to the above, it is also possible to add refreshing agents such as menthol, borneol, camphor, geraniol, eucalyptus oil, bergamot oil, fennel oil, peppermint oil, rose oil, or coolmint, to the ophthalmic solution, for the purpose of providing a refreshing feeling to the eye at the time of administrating the ophthalmic solution.

In certain clinical conditions, the ophthalmic solutions may be formulated with other pharmaceutical agents for the purpose of suppressing discomfort, itch, irritation, or pain in the eye. Such agents may include, but are not limited to, a vasoconstrictor such as phenylephrine, oxymetazoline, naphthazoline, or tetrahydrozoline; a mast-cell stabilizer such as olopatadine; an antihistamine such as azelastine; an antibiotic such as tetracycline; a steroidal anti-inflammatory drug such as betamethasone; a non-steroidal anti-inflammatory drug such as diclofenac; an immunomodulator such as imiquimod or interferons; and antiviral agents such as valaciclovir, cidofovir, or trifluridine.

One way of making the ophthalmic solution is to take the agent and mix it into a liquid with purified water or buffer and adjust, if necessary, for having a pH value within the range of about 5.3 to about 8.5, about 6.0 to about 8.0, or about 7.0 to about 8.0. Examples of buffering agents to maintain or adjust pH include, but are not limited to, citrate buffers, acetate buffers, phosphate buffers, and borate buffers as described above. The ophthalmic solution is also adjusted for isotonicity if needed. Examples of tonicity adjustors are sodium chloride, mannitol, and glycerin as described above. Preferably, the osmotic pressure of the solution is adjusted to be within a range of 200 to 400 mOsm/kg.

The ophthalmic solution can be delivered as eye drops, for example, by instilling drops into the lower eyelid by a drop dispenser. The ophthalmic solution may be used as single dose type eye drops, in which the ophthalmic solution is used off in one administration. Alternatively, the ophthalmic solution can be used as multi dose type eye drops included in a container with a drop dispenser. In such formulations, preservatives may be added to prevent microbial contamination after opening of the container. Such preservatives are typically employed at a level of from 0.001 to about 1.0% weight/volume.

An active agent that is delivered according to the method of the present invention can also be provided in an ophthalmic ointment in an amount that is pharmaceutically effective. Such an ointment can be prepared by mixing the agent with a general ophthalmic ointment base. Examples include purified lanolin, white petrolatum, macrogol, Plastibase, liquid paraffin, and the like. Many of the additive agents described in connection with the ophthalmic solution above can also be optionally added into the ointment. Preferably, the ointment is sterile.

Applications:

The method of the present invention for delivering a targeting nucleic acid to the brain tissue to hybridize a target nucleic acid has many practical applications. In one aspect, it can be used to detect the expression of a gene in the brain tissue by linking a contrast agent to the targeting nucleic acid. If the expression of the gene indicates a disease or disorder in the brain, the method can be used for diagnosing the disease or disorder. For example, the targeting nucleic can have a sequence complementary to a viral nucleic acid such as an RNA transcribed from a viral gene for detecting viral infection in the brain. As another example, brain tumors such as glioma overexpressing certain target genes (e.g., gfap for glioma) can be imaged and detected. Other examples have been provided above. Similarly, the method can be used to detect the expression of a gene introduced into the brain tissue for gene therapy. Likewise, the method can be used to detect stem cells implanted into the brain tissue for therapeutic purposes because stem cells typically have specific patterns of gene expression in comparison to differentiated cells. Therefore, the survival and activity of the stem cells can be monitored without biopsy. Further, the method can be used to monitor the effectiveness of a therapy by monitoring the expression of a target gene which reflects the effectiveness of the therapy.

To detect the hybridization between the targeting nucleic acid and the target nucleic acid, an active agent comprising the targeting nucleic acid and the contrast agent linked together is administered to an eye of a human and non-human animal in an amount sufficient for providing a detectable image and sufficient time is allowed for the active agent to travel to the brain tissue, for the agent to hybridize or bind to the target nucleic acid if the target nucleic acid is present, and for the unhybridized/unbound agent to leave the brain tissue. The brain tissue is then imaged to detect the hybridization of the targeting nucleic acid to the target nucleic acid. It is noted that the active agent may need to be administered multiple times to achieve a detectably effective amount.

In another aspect, the present invention can be used to treat a disease or disorder in the brain by decreasing the expression of a disease gene in the brain. In this regard, an active agent comprising a target nucleic acid complementary to an RNA transcribed from the disease gene (e.g., an antisense nucleic acid, an siRNA, or an shRNA) is administered to an eye of a human or non-human animal having the disease or disorder in an amount sufficient to decrease the expression of the gene and to treat the disease or disorder. For example, the targeting nucleic can have a sequence complementary to an RNA transcribed from a viral gene for treating viral infection. As another example, stroke can be treated by using a targeting nucleic acid complementary to polyADP-ribose polymerase mRNA to inhibit its expression. A contrast agent can be optionally linked to the targeting nucleic acid to monitor the delivery of the targeting nucleic acid to the brain by medical imaging.

In another aspect, the present invention can be used to treat a disease or disorder in the brain by linking a therapeutic agent to a targeting nucleic acid, which delivers the therapeutic agent to the target brain site. In this regard, an active agent comprising the targeting nucleic acid and the therapeutic agent linked together is administered to an eye of a human or non-human animal having the disease or disorder in an amount sufficient to treat the disease or disorder. The targeting nucleic acid is designed to hybridize to a target nucleic acid present at the disease or disorder site so that the therapeutic agent is delivered to the site. For example, the targeting nucleic acid may have a sequence complementary to an RNA transcribed from a viral gene for delivering the therapeutic agent to the viral infection site. As another example, a chemotherapeutic agent can be delivered to brain tumor cells by targeting an mRNA in the tumor cells (e.g., gfap for glioma cells). Other examples have been provided above. In those cases wherein the RNA molecule targeted by the targeting nucleic acid is the transcript of a disease gene, the targeting nucleic acid may also serve a therapeutic function by decreasing the expression of the gene. To monitor the delivery of the therapeutic agent to the brain by medical imaging, a contrast agent can optionally be included in the active agent wherein the contrast agent can be linked to the targeting nucleic acid, the therapeutic agent, or both. A cytotoxic agent can be a useful therapeutic agent for any proliferative diseases or disorders in the brain resulting from excessive or uncontrolled cell growth such as brain tumors. A cytotoxic agent may also be a useful therapeutic agent for viral infection in the brain.

Imaging:

After a sufficient amount of time for an active agent containing an contrast agent to be localized to appropriate brain regions and/or internalized by the appropriate brain cells and for any unbound active agents to leave the brain tissue, the brain tissue is imaged.

Images can be generated by virtue of differences in the spatial distribution of the active agents containing contrast agents which accumulate at a site of tumor, infection, inflammation or other diseases or disorders in the brain. The spatial distribution may be measured using any means suitable for the particular contrast agent, for example, an MRI apparatus, a gamma camera, a PET apparatus, or a SPECT apparatus. Some lesions may be evident when a less intense spot appears within the image, indicating the presence of tissue in which a lower concentration of contrast agent accumulates relative to the concentration of contrast agent which accumulates in surrounding tissue. Alternatively, a lesion may be detectable as a more intense spot within the image, indicating a region of enhanced concentration of the contrast agent at the site of the lesion relative to the concentration of agent which accumulates in surrounding tissue. Accumulation of lower or higher amounts of the contrast agent at a lesion may readily be detected visually. Alternatively, the extent of accumulation of the contrast agent may be quantified using known methods.

MRI can be performed in live animals or humans using standard MRI equipment, e.g., clinical, wide bore, or research oriented small-bore MRI equipment, of various field strengths. Imaging protocols typically consist of T1, T2, and T2* weighted image acquisition, T1 weighted spin echo (SE 300/12), T2 weighted SE (SE 5000/variable TE) and gradient echo (GE 500/variable TE or 500/constant TE/variable flip angles) sequences of a chosen slice orientation at different time points before and after administration of active agent. For example, the brain tissue can be imaged with a series of high-resolution T2*-weighted MR images, e.g., taken 1, 2, or 3 days after an active agent containing a contrast agent is administered.

The active agents containing a contrast agent may shorten the relaxation times of tissues (T1 and/or T2) and produce brightening or darkening (contrast) of MR images of cells, depending on the tissue concentration and the pulse sequence used. In general, with highly T2 weighted pulse sequences and when iron oxides are used, darkening will result. With T1 weighted pulse sequences and when gadolinium chelates are used, brightening will result. Contrast enhancement will result from the selective uptake of the active agent in cells that contain the target gene.

Examples of the present invention are described below. However, the present invention is not in any way limited to the aspects described in the examples.

### Example 1

### Contrast Agent Linked to a Nucleic Acid

An active agent of the present invention may comprise a nucleic acid linked to contrast agent such as an MRI contrast agent (e.g., a magnetic particle) that changes the relaxivity of the cells once internalized so that they can be imaged using MRI. The MRI contrast agent can be a paramagnetic label such as a superparamagnetic iron oxide particle whose maximum diameter is between 1 nm and 2,000 nm (e.g., between 2 nm and 1,000 nm or between 10 nm and 100 nm). The particle can be attached to the nucleic acid through entrapment in a cross-linked dextran. In some embodiments, the particle is a monocrystalline iron oxide nanoparticle (MION), an ultra small superparamagnetic iron oxide particle (USPIO), or a cross-linked iron oxide (CLIO) particle. In some other embodiments, the paramagnetic label is a chelated metal such as Gd³⁺ or Dy³⁺.

One nucleic acid molecule can have multiple (e.g., 2, 3, or more) contrast agent molecules attached (all or some the same or different), or a multiple set of active agents can be created that all have the same nucleic acid and 2 or more different contrast agents. Alternatively, a multiple set of active agents can be made that have different nucleic acids that target different portions of the same target gene (or that target different target genes) and that each have the same or different contrast agents linked thereto.

In one form, the active agent comprises a DNA molecule of 12 to 35 nucleotides (also referred to herein as an oligodeoxynucleotide or ODN), one or more contrast agent molecules, linked to either the 5′ or 3′ ends of the ODN, e.g., by a covalent bond directly or via an optional linker group or “bridge” (e.g., a linkage of a desired length) between the ODN and the contrast agent molecules. The nucleic acid can be either single-stranded DNA or RNA, and is typically an antisense strand, and thus complementary, to a portion of the target nucleic acid to which it hybridizes. The ODN may include one or multiple internal sites that can be attached to a contrast agent, e.g., labeled for example, with a fluorescent or radioactive label.

An active agent can optionally include an antibody that can be attached at either end of the active agent molecule. Such antibodies are typically ones that bind specifically to cell-surface antigens of particular cells or cell types to direct the active agents to the appropriate cells. Once on the surface of the cell, the active agents pass through the cell membrane and into the cells, thereby delivering the contrast into the cell. Once in the cell, the nucleic acids hybridize preferentially to their specific target nucleic acid, such as an mRNA, and remain bound within the cell. Absent the nucleic acid molecule in the active agents, the contrast agents are not retained within the cells.

The nucleic acid can be linked to the contrast agent by a variety of methods, including, e.g., covalent bonds, bifunctional spacers (“bridge”) such as, avidin-biotin coupling, Gd-DOPA-dextran coupling, charge coupling, or other linkers.

The contrast agents can be magnetic particles such as superparamagnetic, ferromagnetic, or paramagnetic particles. Paramagnetic metals (e.g., transition metals such as manganese, iron, chromium, and metals of the lanthanide group such as gadolinium) alter the proton spin relaxation property of the medium around them.

There is considerable latitude in choice of particle size. For example, the particle size can be between 1 nm and 2,000 nm, e.g., between 2 nm and 1,000 rim (e.g., 200 or 300 nm), or between 10 nm and 100 nm, as long as the particles can still be internalized by the cells. The magnetic particles are typically nanoparticles. Preferably, within any particular probe preparation, particle size is controlled, with variation in particle size being limited, e.g., substantially all of the particles having a diameter in the range of about 30 nm to about 50 nm. Particle size can be determined by any of several suitable techniques, e.g., gel filtration or electron microscopy. An individual particle can consist of a single metal oxide crystal or a multiplicity of crystals.

There are two types of contrast agents useful for MRI: T1 and T2 agents. The presence of T1 agents, such as manganese and gadolinium, reduces the longitudinal spin-lattice relaxation time (T1) and results in localized signal enhancement in T1 weighted images. On the other hand, the presence of a strong T2 agent, such as iron, will reduce the spin-spin transverse relaxation time (T2) and results in localized signal reduction in T2 weighted images. Optimal MRI contrast can be achieved via proper administration of contrast agent dosage, designation of acquisition parameters such as repetition time (TR), echo spacing (TE) and RF pulse flip angles.

Specific examples of such magnetic nanoparticles include MIONs as described, e.g., in U.S. Pat. No. 5,492,814; U.S. Pat. No. 4,554,088; U.S. Pat. No. 4,452,773; U.S. Pat. No. 4,827,945; and Toselson et al., Bioconj. Chemistry, 10:186-191 (1999). These particles can also be SPIOs, USPIOs, and CLIO particles (see, e.g., U.S. Pat. No. 5,262,176). SPION (superparamagnetic iron nanoparticles) and nanoparticles employed in the active agents of the present invention should be those that remain in suspension and do not form aggregates in the presence of a magnet.

MIONs can consist of a central 3 nm monocrystalline magnetite-like single crystal core to which are attached an average of twelve 10 kD dextran molecules resulting in an overall size of 20 nm (e.g., as described in U.S. Pat. No. 5,492,814 and in Shen et al., Magnetic Resonance in Medicine, 29:599-604 (1993), to which nucleic acids can be conjugated for targeted delivery.

The dextran/Fe w/w ratio of a MION can be 1.6:1. R1=12.5 mM sec⁻¹, R2=45.1 mM sec⁻¹ (0.47 T, 38° C.). At room temperature relaxivity in an aqueous solution at room temperature and 0.47 Tesla can be approximately 19/mM/sec for R1 and approximately 41/mM/sec for R2. MIONs elute as a single narrow peak by high performance liquid chromatography with a dispersion index of 1.034; the median MION particle diameter (of about 21 nm as measured by laser light scattering) corresponds in size to a protein with a mass of 775 kD and contains an average of 2,064 iron molecules.

The physicochemical and biological properties of the magnetic particles can be improved by crosslinking the dextran coating of magnetic nanoparticles to form CLIOs to increase blood half life and stability of the contrast agent complex. The crosslinked dextran coating cages the iron oxide crystal, minimizing opsonization. Furthermore, this technology allows for slightly larger iron cores during initial synthesis, which improves the R2 relaxivity. CLIOs can be synthesized by crosslinking the dextran coating of generic iron oxide particles (e.g., as described in U.S. Pat. No. 4,492,814) with epibromobydrin to yield CLIOs as described an U.S. Pat. No. 5,262,176.

The magnetic particles can have a relaxivity on the order of 35 to 40 mM/sec, but this characteristic depends upon the sensitivity and the field strength of the MR imaging device. The relaxivities of the different active agents can be calculated as the slopes of the curves of 1/T1 and 1/T2 vs. iron concentration; T1 and T2 relaxation times are determined under the same field strength, as the results of linear fitting of signal intensities from serial acquisition of (1) inversion-recovery MR scans of incremental inversion time for T1 and (2) SE scans of a fixed TR and incremental TE. Stability of the conjugates can be tested by treating them under different storage conditions (4° C., 21° C., and 37° C. for different periods of time) and performing HPLC analysis of aliquots as well as binding studies.

In some embodiments, the paramagnetic label on the probe is a metal chelate. Suitable chelating moieties include macrocyclic chelators such as 1,4,7,10-tetrazazcyclo-dodecane-N,N′,N″,N′″-tetraacetic acid (DOTA). For in vivo use, e.g., as MR contrast agents in a human patient, gadolinium (Gd³⁺), dysprosium (Dy³⁺), and europium are suitable. In other embodiments, CEST (Chemical Exchange Saturation Transfer) can be used. The CEST method uses endogenous compounds such as primary amines as contrast agents that can be linked to the ODN.

Other suitable contrast agents are labels such as near infrared molecules, e.g., indocyanine green (ICG) and Cy5.5 and quantum dots, which can be linked to the nucleic acid and used in optical imaging techniques, such as diffuse optical tomography (DOT) (see, e.g., Ntziachristos et al., Proc. Natl. Acad. Sci. USA, 97:2767-2773, 2000). Fluorescent labels, such as FITCs, Texas Red, and Rhodamine can also be linked to the nucleic acid. Radionuclides, such as ¹¹C, ¹³N or ¹⁵O, can be synthesized into the nucleic acids to form the active agents. In addition, various known radiopharmaceuticals such as radiolabeled tamoxifen (used, e.g., for breast cancer chemotherapy) and radiolabeled antibodies can be used. Such particles can be coated with dextran for attachment to the nucleic acids as described herein. These radio-conjugates have application in PET. Radioisotopes, such as ³²P, ³³P, ³⁵S (short half-life isotopes) (Liu et al. Ann. Neurol., 36:566-576, 1994), radioactive iodine, and barium can also be integrated into or linked to the nucleic acid to form active agents that can be imaged using X-ray technology.

Note that two or more contrast agent molecules, of the same or different kinds, can be linked to a single nucleic acid.

The nucleic acids are typically single-stranded, anti-sense oligonucleotides of 12, 15, 18, 20, 23, 25, 26, 30 and up to 35 nucleotides in length. They are designed to hybridize to the target gene (if present in sufficient numbers in a cell), or to hybridize to a messenger RNA transcribed from the gene whose expression is to be imaged. They can be protected against degradation, e.g., by using phosphorothioate, which can be included during synthesis. In addition, by keeping the length to 35 or fewer nucleotides (preferably 30 or fewer nucleotides), the non-specific nuclease/protease response that could destroy cellular mRNA and induce a cytotoxic reaction can be minimized.

The contrast agent and the nucleic acid are linked to produce the active agent using any of several known methods. For example, if the contrast agent is a MION, this molecule can be linked to a nucleic acid by phosphorothioating the oligonucleotide and labeling it with biotin at the 5′ end. The dextran coated MION can be activated and conjugated to the biotin-labeled oligonucleotide using avidin based linkers, such as NeutrAvidin (Pierce Chem.).

In addition, liposomes, lipofectin, and lipofectamine can be used to help get the entire active agent into a cell.

### Example 2

### Delivery of Nanoparticles with a Dual Function of Imaging and Targeting Gliosis to Targeted Brain Cells Via Eye Drops

Brain injury affects one-third of survivors of heart attack. Detecting afflicted tissue by magnetic resonance imaging (MRI), however small and inaccessible by biopsy, can be useful for improving prognosis and correlating behavior deficits. We developed a modular MR probe targeting the gene transcript of glial fibrillary acidic protein (sODN-gfap) to detect glia, known to form scar tissue of the brain. We demonstrate in this example that this probe detected gliosis of brain injury by puncher wound or cerebral ischemia, after application from an eye dropper to the conjunctival sac of C57Black6 mice. This type of modular has other clinical applications such as non-invasive targeting of gene actions in different brain cells when specific mRNA transcripts are known to impact a specific neurological disorder.

### Materials and Methods

SPION-NeutrAvidin: SPION was prepared and purified for these studies in the A. A. Martinos Center for Biomedical Imaging at Massachusetts General Hospital (MGH), as described in Shen T et al., Magn Reson Med 29, 599-604, 1993; and Lind K et al., J Drug Target 10, 221-230, 2002. Freshly synthesized SPION in 1.5N NaOH solution was functionalized with chloroethylamine (2 M), and linked to NeutrAvidin (NA) in the presence of 1 M sodium cyanoborohydride (both from Pierce Biotechnology, Rockford, Ill.). The resulting covalently linked product, SPION-NA, was filtered and dialyzed against a 20× volume of sodium citrate buffer solution (25 mM, pH 8.0), using a Centricon Plus-100 filter (100 KD cut-off, Millipore Corp., Bedford, Mass.). Activated SPION (SPION-NA) was stored in an amber-colored bottle at 4° C., at a concentration of 4 mg iron per ml sodium citrate buffer. Iron concentrations in SPION samples were determined by optical absorbance at 410 nm after treatment with hydrogen peroxide (0.03%) and 6N hydrogen chloride (de Marco G et al., Radiology 208, 65-71, 1998).

Conjugation of Biotinylated sODN to SPION-NA: We synthesized three 5′-biotin-labeled antisense sODN: sODN-gfap, 5′-gtctccgctccatcctgccc-3′, SEQ ID NO:1; sODN-actin, 5′acgcagctcagtaacagtccgccta-3′, SEQ ID NO:2 (Schedlich L et al., Biol Cell 89, 113-122, 1997); and sODN-Ran, a randomized sequence, as negative control, 5′-gggatcgttcagagtctag-3′, SEQ ID NO:3 (Zhang Y M et al., J Nucl Med 42, 1660-1669, 2001). Single-stranded ODNs were protected from non-specific nuclease using phosphorothioation in all nucleotide bridges. All sODN were purified using polyacrylamide gel electrophoresis (PAGE). To directly observe the sODN, we also synthesized sODN with fluorescein isothiocyanate (FITC) on the 5′ terminus and biotin on the 3′ terminus (FITC-sODN-biotin). SPION-NA (250 nmol Fe) was incubated with the biotinylated sODN (FITC-sODN-biotin, 1 nmol) at room temperature for 30 minutes and the mixture was filter-dialyzed with 3 washes of sodium citrate buffer (25 mM, pH 8) in a centrifugal filter device (Microcon YM-30, Millipore). We incubated unconjugated SPION of the same lot from which SPION-sODN had been made with saline. The probes were re-suspended in 36 μl sodium citrate buffer. After conjugation, the probes were stored at 4° C. (no longer than 24 hours). Immediately before administration, we optionally added 4 μA of lipofectin (0.1 mg/ml, Invitrogen Life Technologies).

Delivery: SPION-sODN in 25 mM sodium citrate (pH 8) was delivered at 0.1 mL via an eye drop solution at 10 μl to each eye per application and repeated every 15 minutes for 5 applications till a total of 0.1 mL was administered. Each animal received 4 mg Fe per Kg. On day later, the uptake/retention of SPION-sODN was monitored by MRI.

In vivo Data Acquisition: In vivo image acquisition was performed using a 9.4 Tesla MRI scanner (Bruker Avance system, Bruker Biospin MRI, Inc.) at one day after ophthalmic delivery of SPION. Animals were anesthetized with pure O₂ and 2% halothane (800 ml/min flow rate). We positioned a custom-built 1-cm transmit/receive surface coil on the heads of the animals, which were placed in the prone position in a home-built cradle. Gradient echo (GE) images of constant repetition time (TR) and incremental echo spacing (TE) were acquired along the axial direction. Acquisition parameters were as follows: TR=500 ms, TE=3, 4, 6, 8 and 10 ms, flip angle=30, 1.0 mm or 0.5 mm slices, 15×15 mm field of view (FOV); 128×128 pixel T₂* maps were calculated using a pixel-wise linear fitting algorithm on the series of images. To compare elevation of mRNA, we constructed R₂* maps (the inverse of T₂* maps), which showed signal elevation with SPION retention.

Data analysis: We selected MR slices from MR data of each animal group for t-test statistical analysis, but avoided those with partial volume artifacts in regions close to the olfactory bulbs and the nose. We used in-house software (Martinos Center for Biomedical Imaging at Massachusetts General Hospital) to co-register the images of whole mouse brains, then superimposed and averaged the R₂* maps within designated brain slices. A computer-generated scale is also included in the R₂* maps.

BCAO animal model: Brain damage after heart attack is predictive of poor neurological prognosis (R. O. Roine et al., Stroke 24, 1005, 1993; M. C. Geraghty and M. T. Torbey, Neurol Clin 24, 107, 2006; and M. Fujioka et al., Stroke 25, 2091, 1994). Cerebral ischemia resulting from the deposition of microemboli in the brain's fine vasculature is one important cause of brain lesion; brain injury after cardiac surgery is likewise an important contributing factor in morbidity and mortality. Abnormal water content or gross morphological changes resulting from transient metabolic disturbance and brain edema may be identified by hyperintensity in diffusion-weighted imaging (hDWI) and significant reduction in the apparent diffusion coefficient (ADC) in the brain using MRI (A. Bizzi et al., AJNR Am J Neuroradiol 14, 1347, 1993; and M. E. Moseley et al., Stroke 24 (12 Suppl), 160, 1993). It has been observed that C57Black6 mice treated with bilateral occlusion of the common carotid arteries (BCAO) for 60 minutes develop hDWI resulting from cerebral ischemia that simulates metabolic disturbance (Liu C H et al., FASEB J 2007, in press, E-publication: May 30, 2007). The mortality rate for C57Black6 mice treated with 60-minute BCAO is 20% (N=10) if lactated Ringers plus dextrose (5%, 1 ml subcutaneously) is given daily during the first week after the episode. We have detected brain injury in this model by Evans blue extravasations for leakage in the blood-brain barrier (BBB) at the beginning of reperfusion or terminal UTP nick-end labeling (TUNEL) for DNA fragmentation at one day of reperfusion. This model induces brain damage in the cortex, hippocampus, striatum and the nigrostiatal bundle (tissue near the substantia nigra) by these two assays. Though effective, both assays rely on the use of postmortem samples, and these assays thus limited in their translatability to living subjects.

### Results

Gliosis is a process involving the outgrowth of a fibrous network of glia in the region of damage; this outgrowth of glia, though a normal repair process to brain damage, leads to scar formation in the central nervous system, and is a permanent feature of many human neurological disorders including, but not limiting to brain tumor, viral encephalitis, multiple sclerosis and stroke. Gliosis is associated with many neurological disorders, and is especially characteristic of tumor formation and repair of injury caused by traumatic brain injury, stroke, or cardiac arrest (C. H. Liu et al., J Neurosci 27, 713, 2007; H. E. Killer et al., J Neuroopthalmol 19, 222, 1999; A. J. Dickinson and R. E. Gausas, Eye 20, 1145, 2006). Cerebral ischemia produces a gliotic reaction by elevating glial fibrillary acidic protein (GFAP) immunochemistry at the site of infarction and/or neuronal death (C. Chiamulera et al., Brain Res 606, 251, 1993; T. Fahrig, J Neurochem 63, 1796, 1994; and C. H. Liu et al., Mol Imaging 6, 156, 2007). Gliosis or the expression of inhibitory molecules from scarring glia following CNS injury, reduces neurite outgrowth (R. J. McKeon et al., J Neurosci 11, 3398, 1991; M. Nieto-Sampedro, Adv Exp Med Biol 468, 207, 1999; R. J. Gilbert et al., Mol Cell Neurosci 29, 545, 2005; I. Rozovsky et al., Neurobiol Aging 26, 705, 2005).

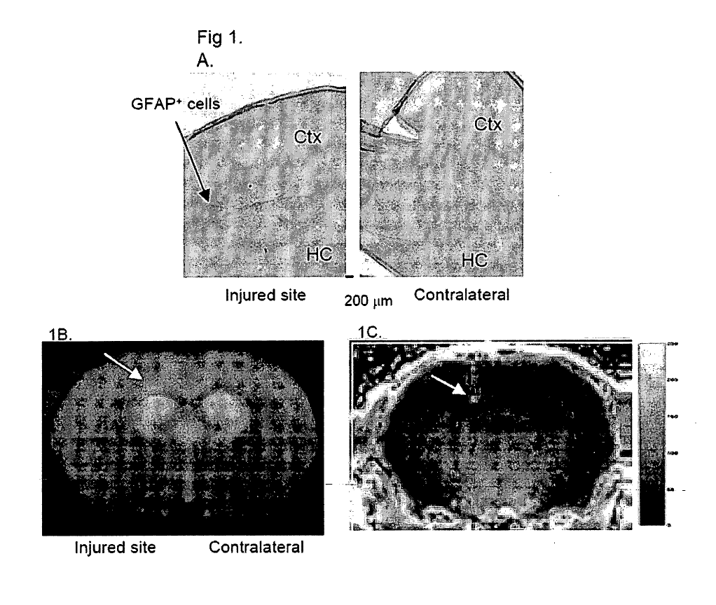

To provide an MRI protocol for the detection of small brain damage in the brain, we chose cells that express glial fibrillary acidic protein (GFAP) because reactive glia are present at the site of DNA fragmentation. Our hypothesis is based on reports that neuronal loss and elevated glia (gliosis) are found at the injured site of the brain after cardiac arrest (C. H. Liu et al., J Neurosci 27, 713, 2007). To detect gliosis at the site of injury (C. H. Liu et al., J Neurosci 27, 713, 2007; H. E. Killer et al., J Neuroopthalmol 19, 222, 1999; A. J. Dickinson and R. E. Gausas, Eye 20, 1145, 2006), we first tested whether a short DNA with sequence complementary to mRNA of GFAP (sODN-gfap) would report gliosis in the brain using the orbital delivery. We induced brain injury by intracranial puncture in the left hemisphere of six animals; three of them were sacrificed three days later for evidence of gliosis around the injured site (FIG. 1A). In the remaining three animals, we observed no remarkable lesion in T2-weighted MRI in the left cortex compared to the right cortex eight weeks later (FIG. 1B). We conjugated SPION-NeutrAvidin with biotinylated sODN-gfap. One day after SPION-gfap delivery by an eye dropper, we acquired R2* maps because R2* value is positively correlated with intracellular iron oxide in mouse brain (T. M. Ringer et al., Stroke 32, 2362, 2001). We observed regions with hyperintense R2* values at the injured site (arrows, FIG. 1C). R2* hyperintensity is consistent with immunohistochemistry shown in FIG. 1A, which shows elevations of GFAP-positive cells in the tissue surrounding the injured site in the left (ipsilateral) hemisphere but normal patterns of GFAP-positive cells in the right (contralateral) hemisphere. One week after MRI, hyperintense R2* maps are no longer detectable, suggesting removal of the probes (T. M. Ringer et al., Stroke 32, 2362, 2001). Our data presented here show that SPION-gfap can be delivered non-invasively via eye drops to a cohort of brain cells that express gfap mRNA.

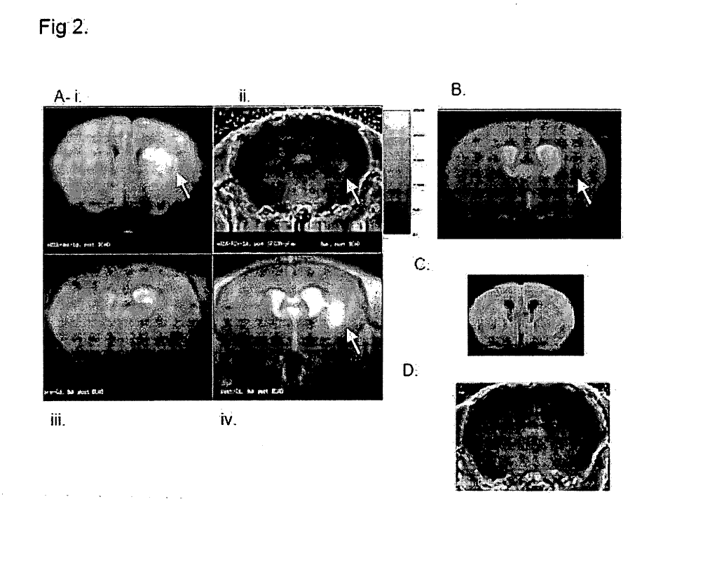

To illustrate the application of this probe for detection of gliosis after neurological disorders in living subjects, we investigated gliosis reporting using SPION-gfap and ocular delivery in two groups of animals receiving BCAO (N=8) (Liu C H et al., FASEB J 2007, in press, E-publication: May 30, 2007; and Liu C H et al., Mol Imaging, 6, 156-170, 2007) and sham operation (the same surgical procedure without BCAO, N=4). MRI for hDWI/rADC (hyperintensity in diffusion-weighted imaging/reduction in the apparent diffusion coefficient) obtained at one day post-BCAO showed metabolic disturbance in expanded areas of the striatum of seven animals, and the midbrain of one animal (one representative animal is shown in panel i of FIG. 2A). Eight weeks after BCAO, we observed no obvious injury site in the T2-weighted MR images in all mice, although certain regions did show possible injury by comparison to the hDWI MRI (arrow in FIG. 2B). After delivering SPION-gfap in an eye drop solution and allowing one day for uptake and distribution, we found hyperintense R2* maps in the brains of all seven animals (one is shown in panel ii of FIG. 2A), whose BBB leakage is validated using Gd-DTPA (diethylenetriamine pentaacetic acid gadolinium, 0.4 mM/kg, intravenously) one week later (panels iii and iv of FIG. 2A). The region that showed diffused Gd entrapment in panel iv of FIG. 2A is within the region that exhibited hDWI during early reperfusion (FIG. 2Ai), and overlaps with the regions of hyperintense R2* maps (FIG. 2A ii). On the other hand, no swelling or hDWI was observed in all of the sham-operated (100%) animals (FIG. 2C). We observed no anomalies from R2* map in the entire brain after similar SPION-gfap application (FIG. 2D). Among the matching locations for hDWI, the R2* maps and BBB leakage the lesion detected by SPION-gfap is the most focused one and is located within the region that show hDWI and BBB leakage. These studies demonstrate that gliosis can be detected non-invasively using SPION-gfap and eye drop delivery in live subjects after cerebral ischemia.

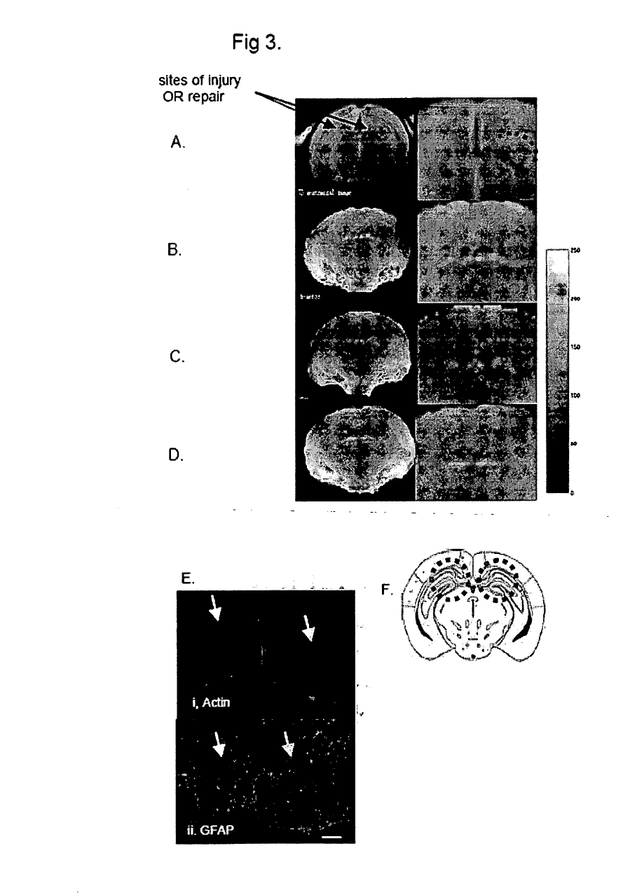

In a second series of experiments, we tested whether other antigens can be expressed at the site of brain injury. We applied SPION-actin, SPION-gfap and SPION-mmp-9 serially to the same animals previously treated with BCAO of 60 minutes. We first acquired T2W images and selected 4 animals with scar tissue in T2W MRI (one representative animal is shown in FIG. 3A); we applied SPION-sODN at the time and acquired SPION retention the next day in all animals. We observed elevated SPION-actin uptake throughout the brain, with specific focal retention ventral to the injured dentate gyms of the hippocampus (arrows, FIG. 3B).

The same is observed for SPION-gfap which has been applied a few weeks later. Focal retention of SPION-gfap is present at the injured sites (arrows, FIG. 3C). SPION-mmp-9 reported cells that express mmp-9 are located in the cells expressing actin mRNA three weeks earlier. FIG. 3D shows actin-expressing cells are located in the ventricle and GFAP-expressing cells (gliosis) becomes dominant feature at the location where R2* value elevated in the R2* maps of tMRI. Our immunohistochemistry at the hippocampus supports results shown in tMRI using SPION-actin and SPION-gfap. Because gliosis has been reported in injured site of the brain, our observation of focal SPION retention after SPION-gfap is consistent with gliosis after cerebral ischemia. The mechanism of focal SPION-actin and SPION-mmp-9 retention in the same cells may suggest vascular-remodeling during brain repair. Actin is expressed during skin scarring if no pressure is applied (A. M. Costa et al., The American journal of pathology 155, 1671, 1999), and actin is one of many genes expressed in CNS microvascular pericytes with multipotential stem cell activity (P. Dore-Duffy et al., J Cereb Blood Flow Metab 26, 613, 2006). Our in vivo observation supports brain repair as one characteristic feature to BCAO induced brain injury. This experiment demonstrates that ocular delivery can be used for multiple probes imaging through time.

Therefore, this study shows that the specificity and the route of non-invasive eye delivery of SPION-gfap can assist future diagnosis of gliosis in the brain in living subject with clinical applications, not only for detection of brain scarring after cerebral ischemia, but also for small regions of gliosis or gene expression that may be glial specific.

Moreover, this modular probe can be cleared from the brain within three days after tMRI. This will allow repetitive applications in the same subject using the same or different probe. Postmortem samplings in conventional molecular biology assays not only terminate monitoring in biomedical research, but also remove tissue that may be worthy of saving for efficacy analysis. The availability of this method and novel contrast probe with a dual function of imaging and targeting will enable real-time investigation on wound healing in the central nervous system.

### Example 3

### BBB Leakage Induced by Cortical Spreading Depression (CSD) Allows SPION-sODN Uptake in the Brain Via Eye Drop Delivery

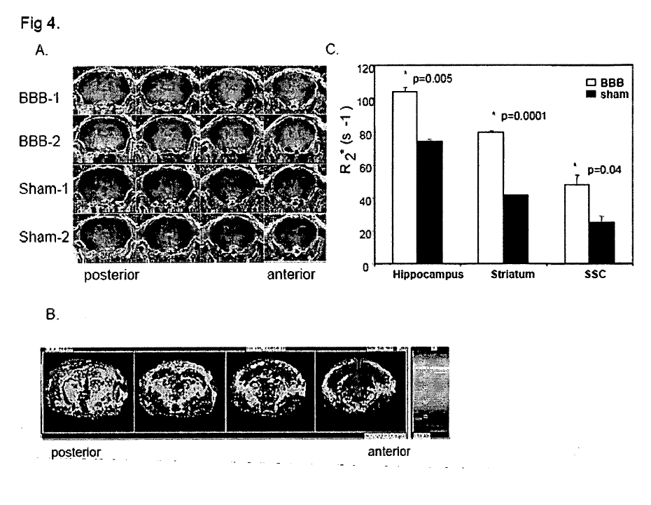

We induced BBB leakage by cortical spreading depression in two mice. This model does not induce brain injury in the hippocampus. Using methods as described in Example 2, we delivered SPION-actin-FITC to these two mice and two sham-operated mice via eye drops. SPION-R2* maps were obtained and analyzed (FIGS. 4A-4C).

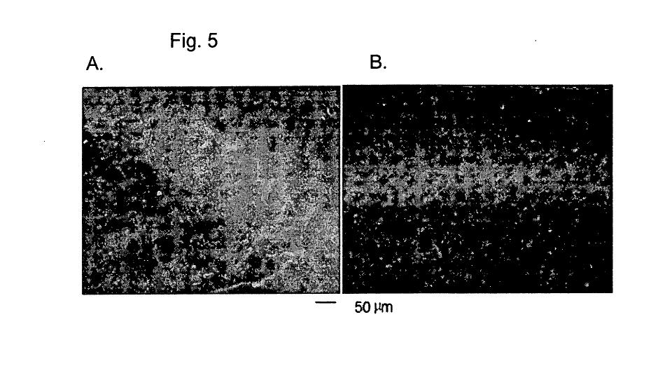

Histology analysis of the CA1 neuronal formation of the hippocampus in the CSD and sham-operated animals was also conducted after eye delivery of SPION-actin-FITC (FIGS. 5A and 5B). Samples were stained red with murine monoclonal Texas Red-IgG to provide enhanced contrast for actin-FITC (yellow).

We observed that animals having BBB leakage, but not sham-operated animals, retained SPION-actin in the brain cells that express actin mRNA.

### Example 4

### MR Contrast Probe that Reports Amphetamine-Induced Gene Transcription In Vivo

To determine whether gene transcription can be related to amphetamine exposure, we determine the elevation of mRNA transcripts in live C57black6 mouse brains. We infused superparamagnetic iron oxide nanoparticles (SPION), a MR T₂* susceptibility agent (SPION=84 pmol per kg) conjugated with phosphorothioate-modified oligodeoxynucleotides (sODN) to cfos, fosB and ΔfosB mRNA [SPION-cfos (5′-catcatggtcgtggtttgggcaaagc-3′OH), SEQ ID NO:4; SPION-fosB (5′-ccttagcggatgttgaccctgg-3′OH), SEQ ID NO:5; and SPION-ΔfosB (5′-acttgaacttcactcggccagcgg-3′OH), SEQ ID NO:6] via the intracerebroventricular (ICV) route to the left cerebral ventricle of animals. The sODN have sequence complementary to and binds to its mRNA specifically. When challenged with amphetamine (4 mg/kg, n=6) to naive animals, the R₂* values (i.e., 1/T₂*) of SPION-cfos and SPION-fosB significantly increased (p<0.05, t test), compared to saline (n=4) in key brain regions responsible for reward, motivation and addiction (the nucleus accumbence, and middle prefrontal cortex), where elevated Fos and FosB have been demonstrated. On the other hand, the retention of SPION-ΔfosB was significantly elevated in animals that have prior exposures to amphetamine (sensitized). Acupuncture treatment after sensitization prevents the elevation of amphetamine-induced elevation in the retention of SPION-ΔfosB (n=4). The example shows that transcription MRI is a sensitive way to validate gene activities in live animals and gene activation after amphetamine begins at the transcription level. Although SPION-sODN agents were administered via the ICV route in this example, they can also be administered via the ophthalmic route along with the transient induction of BBB leakage such as by osmotic shock (manitol 1.6M, intravenous infusion).

Other drugs are useful in anesthetic practice because they produce sedation, amnesia and profound analgesia. However, their use have been limited because they have the potential for postoperative hallucinations, dissociative reactions and delayed recovery. These drugs are known to alter gene action in neurons (mRNA of Fos superfamily). The method of the present invention has applications in drug abuse studies, therapy to drug abuser, assisting in developing a better drug for combating weigh gain without being addition to drug, and comparing gene action in different drug of abuse. These applications can also use ocular delivery of the probe with the use of manitol to open BBB of the brain.

### Introduction

Amphetamine was synthesized in 1887 and its derivatives were used to treat nasal congestion in 1932, prescribed for a sleep disorder (narcolepsy) and attention deficit hyperactivity disorder (ADHD) in 1937. Amphetamine was given to American servicemen to prevent fatigue during the Second World War. Now it is one of the best-known reinforcing psychostimulants abused by humans and is a major health issue in the clinical and scientific communities. Prolonged and repetitive usage of amphetamine can result in psychosis similar to schizophrenia in human subjects. Chronic use appears to result in reduced levels of dopamine, and symptoms like those of Parkinson's disease, has been linked to manic-like symptoms in bipolar disorder. Neurotoxic effect in animals with chronic exposure does not show neuronal death, but show shrinkage in nerve terminals and regrowth is limited. Similarly, repeated amphetamine exposure induces behavior sensitization in animals similar to the drug dependence, psychoses and chronic schizophrenia in humans. The consistent pattern of behavioral changes produced by amphetamine in animals similar to psychosis in human has suggested that these drug-induces changes in animals may provide a model of the endogenous psychosis in humans. Recurrent drug use causes long-lasting neuronal adaptations in the brain that lead to compulsive addictive behavior in humans and in animal models of sensitization; these effects resulting from psychostimulant exposure can be a result of molecular adaptations that alter normal brain function. Many such adaptations have been shown to result from altered regulation of protein phosphorylation and activation of gene transcription of immediate early genes. Changes in gene expression in animals with exposure to amphetamine are different in naive and sensitized animals, and addiction is related to prolonged elevation of deltaFosB (ΔFosB) protein, which is a truncated FosB protein formed by alternative splicing of 140 basepairs from fosB mRNA. The elevation of ΔFosB protein lasts for more than seven days, in contrast to the transient elevation of other members of the Fos family of transcription factors (c-Fos and FosB) and Fos related antigens (Fra-1 and Fra-2). Although elevations of different members of the super family of Fos gene transcripts have been reported after amphetamine application to naive rats, the prolonged elevation of ΔfosB mRNA after amphetamine has not been definitively demonstrated, partially due to the low level of expression and the need to amplification for detection.

### Materials and Methods

The MR Probe: For our MR studies, we generated SPION-fosb and SPION-Δfosb, in addition to SPION-Ran and SPION-cfos which reports the expression of c-fos mRNA. Biotinylated sODN was conjugated to SPION-NeutrAvidin (NA) via an Avidin-biotin linkage. The transverse relaxivities (r2) of SPION-NA and SPION-cfos ranged between 18 to 25 mM⁻¹s⁻¹; these result suggests the addition of a small molecular weight sODN to SPION-NA did not cause significant relaxivity change. Contrast probe preparation including preparation of SPION-NeutrAvidin, conjugation of biotinylated s-ODN to SPION-NA, characterization of SPION-dODN in vitro, delivery of the contrast conjugates (84 pmol SPION-sODN per kg), and induction of mRNA transcription using amphetamine were conducted as described in Liu C H et al., J Neurosci 27, 713-722, 2007, except animals were anesthetized with pure O₂ plus 2% halothane (800 ml/min flow rate) during ICV delivery.

Delivery of Contrast Conjugates: All procedures and animal care practices adhered strictly to AAALAC, Society for Neuroscience, and institutional guidelines for experimental animal health, safety, and comfort. Male C57b6 mice (24±3 g, Taconic Farm) were never exposed to amphetamine (naive), or repeatedly given amphetamine (4 mg/kg, i.p., sensitized) or saline (vehicle) every other day for 14 days. The sensitized animals were withdrawn from amphetamine for 14 days. On the day of MR acquisition, mice were anesthetized (pure O₂ plus 2% halothane (800 ml/min flow rate) and MRI contrast probes (SPION-fosB, SPION-ΔfosB, SPION-cfos [positive control], SPION-Ran [negative control]) were delivery via intracerebroventricular (ICV) route as described in Liu C H et al., J Neurosci 27, 713-722, 2007. Amphetamine (4 mg/kg) or saline vehicle was given (i.p.) four hours later. MRI was acquired three hours after drug administration. At the end of MRI, body weights were taken and recorded and postmortem brain samples were obtained for immunohistology of probe uptake.

In vivo MRI studies: All in vivo animal MRI acquisitions were performed using a 9.4 Tesla MRI scanner (Bruker-Avance system). Animals were anesthetized with pure O₂ plus 2% halothane (800 ml/min flow rate). All image sets were acquired in a custom-built one-cm transmit/receive surface coil was positioned on the head of animals. Animals were placed prone in a home-built cradle. The MRI scanning protocols at each time point were multi-slice gradient echo (GE) imaging sequences (TR=500 ms, TE=2.3, 3, 4, 6, 8 and 10 ms, flip angle=30, 128×128 pixels, 0.5 mm slice, 20 slices, 15 mm FOV, 4 averages) along the axial and sagittal planes. Image analysis was performed using MRVision (MRVision Co, Winchester, Mass.), MATLAB and an in-house software. R₂* maps were constructed and the regional R2* enhancement was related to local MION concentration (Boxerman J L et al. Magn Reson Med 34, 555-566, 1995; and Hamberg L M et al. Magn Reson Med 35, 168-173, 1996).

To identify regions that have SPION-retention above the baseline, we developed R₂* map subtraction by using two-step R₂* map construction (subtraction hotspot identification) (Liu C H et al. FASEB, in press, E-publication: May 30, 2007). The hotspots were identified using the reference to the infusion site and stereotaxic coordinates of C57black6 mouse brain (Paxinos G. and Franklin K. B. J. (2001) The Mouse Brain in Stereotoxic Coordinates, Academic Press Limited, London). To evaluate the level of gene transcription, we selected regions of interest (ROI) in the contralateral hemisphere (brain slices ranging −1.7 to +3.7 mm, bregma) using the infusion site as the reference for alignment; we calculated the mean R₂* values in these ROIs. Average of R₂* values and the standard errors from each of the three groups were obtained and analyzed statistically.

Postmortem tissue preparation: Animals were anesthetized (pure O₂ plus 2% halothane (800 ml/min flow rate) and transcardially perfused with 15 ml heparinized saline at a rate of 10 ml/min, followed by 15 ml of freshly prepared paraformaldehyde (PFA, 4%) in 0.1 M phosphate buffer saline (PBS, pH 7.4) at a rate of 10 ml/min. The brain was removed from the skull and kept in the PFA solution overnight at 4° C., followed by chase, and storage in 20% sucrose/PBS solution. The brains were immediately processed and tissue sections of 50 μm were prepared.

Histological and immunohistochemical staining: The distribution of fluorescien isothiocyanate (FITC)-sODN was examined after counter-stained with mouse anti neurin IgG-TexasRed (Roche Molecular Biochemicals, Pennsburg, Germany). Microscopic images were captured using a SPOT camera (Diagnostic Instruments, Detroit, Mich.).

Statistical Tests: In quantitative studies, as soon as we obtained the first set of data, we calculated the number of animals in each group that was required to achieve 95% power for a p value of 0.01, according to an in-house software (La Morte, “Sample Size Calculations” http://is.partners.org/aniweb/samplesz.doc). Unless power calculation called for a greater number of animals, each study was repeated with at least three animals in each treatment group. When the minimum number of animals is less than 10, we either revised the hypothesis and the protocol. When the number is greater than 10, we determined it “not significant different”.

When data were acquired after the minimum number of animals had been studied, we computed the mean and standard error of the mean (SEM) from the averaged values in each group of animals, and compared the statistical significance of these values using at test (one tail, type II or equal variant, GraphPad Prism IV, GraphPad Software, Inc, San Diego, Calif.). A p value of less than 0.05 is statistically significant.

### Results

Selecting surface coil for transcription MRI: We aimed to reduce the signal drop-off due to a surface coil to better characterize the R₂* elevation. We therefore compare two surface coils (1 cm and 2 cm in diameter) to see if a more uniform excitation profiles can reduce the signal drop off in the lower portion of the brain. Same MRI sequence and geometry were used except that we needed more averaging (NA=4) for the 2 cm coil due to poor SNR (signal to noise ratio). As expected, the signal drop for the rat coil is 50% and the signal drop for the 1 cm coil is 70%. However, as we compared the corresponding R₂* maps, the anatomical clarity was better using the 1 cm coil. We suspected that there may be an overestimation of R₂* in the lower portion of the brain due to signal drop as well as the endogenous iron concentration. This can be overcome by including pre-infusion baseline and as many positive and negative controls in our studies.

Selecting MRI sequence of acquisitions: We compared some of the MRI sequences that can be used in this investigation: (1) Gradient Echo Fast Imaging (GEFI): TR/TEs=500/3, 4, 6, 8, 10 ms, FOV 1.5 cm, 128×128 pixels, slice thickness 0.5 mm, NA=2. (2) Spin Echo (RARE): TR/TEs=7000/30, 45, 60 ms, same geometry as (1), RARE factor=8, NA=2. (3) Echo Planar Imaging (EPI): TR/TE=4000/18 ms, FOV=2.8 cm, 96×128 pixels, slice thickness 0.5 mm, slice separation 1 mm, NR=2. We calculated R₂ maps using sequence (2) and found that R₂ was not a sensitive parameter to represent SPION-sODN retention, although this sequence yielded images with good contrast. We also tried EPI sequence and found that the image distortion was not acceptable due to the brain size. Sequence (1) is the one we have used in all of our studies that was sensitive to detect SPION-sODN using the calculated R₂* maps.

Choice of T2 contrast agent: Several commercial sources of T2 (CST2) contrast agents were used to test for their suitability in making contrast probes for transcription MRI. SPION (0.01 mg/ml) has a property of remaining in suspension when it is being placed to magnetic separation column, while CST2 is attracted to magnet. This property of SPION remains the same after activation to SPION-NeutrAvidin, but remained for one to two days after linking to sODN. Uptakes/distributions at seven hours after ICV delivery of SPION-fosB were determined, and no distribution was observed for CST2-fosB. MION-fosB was entrapped in the ventricular space and produce blooming effect in regions adjacent to the ventricle. We determined that SPION conjugates are most suitable for tMRI.

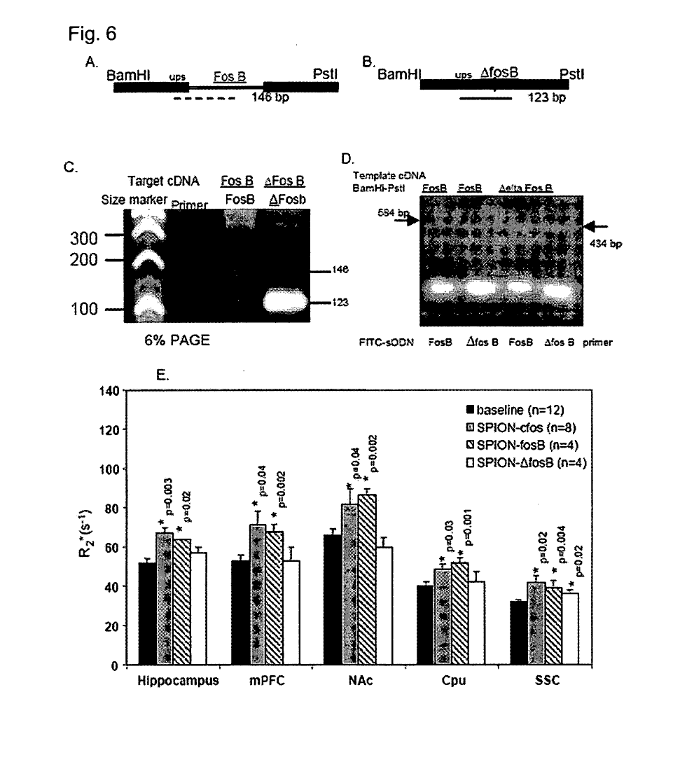

Specificity of SPION-fosB and DPION-ΔfosB on targeting its cDNA: FIG. 6 shows the design of sODN-fosB and sODN-ΔfosB (panels A & B) and the specificity of each sODN on supporting polymerase chain reaction (PCR) to its fragment lengths as predicted from its relative location in the cDNA (panel C). Fluorescein isothiocyanate (FITC) labeled sODN-fosB and FITC-sODN-ΔfosB specifically bind to its cDNA at physiological temperature (panel D). Most important of all, sODN-fosB does not bind to ΔfosB cDNA; sODN-ΔfosB to fosB cDNA.

MR Sensitivity of SPION-sODN: We evaluated MR sensitivity of SPION-fosB and SPION-ΔfosB using serial MRI scans on live mouse brains in the 9.4 Tesla system. We measured and compared mean R₂* values (R₂*=1/T₂*) in selected regions of interest (ROI) in the hemisphere contralateral to the site of infusion. The peak retention profile for SPION-sODN of sODN-cfos, sODN-fosB and sODN-ΔfosB is 7 hours. Regional SPION-retention after ICV infusion of SPION-fosB and SPION-ΔfosB using SPION-cfos as control is shown in FIG. 6E. We observed the significant elevation of SPION retention in the animal that received SPION-cfos and SPION-fosB. The retention profile for SPION-cfos and SPION-fosB is the same. The result indicates c-fos and fosB mRNA, but not ΔfosB mRNA, in the normal brains. The specificity of SPION-ΔfosB is shown by a lack of significant retention in all ROI, except the somatosensory cortex (SSC), in the group that received SPION-ΔfosB. The data indicates endogenous SPION-fosB and SPION-ΔfosB are significantly different in vivo, therefore, SPION-ΔfosB are specific and do not cross binding fosB mRNA.

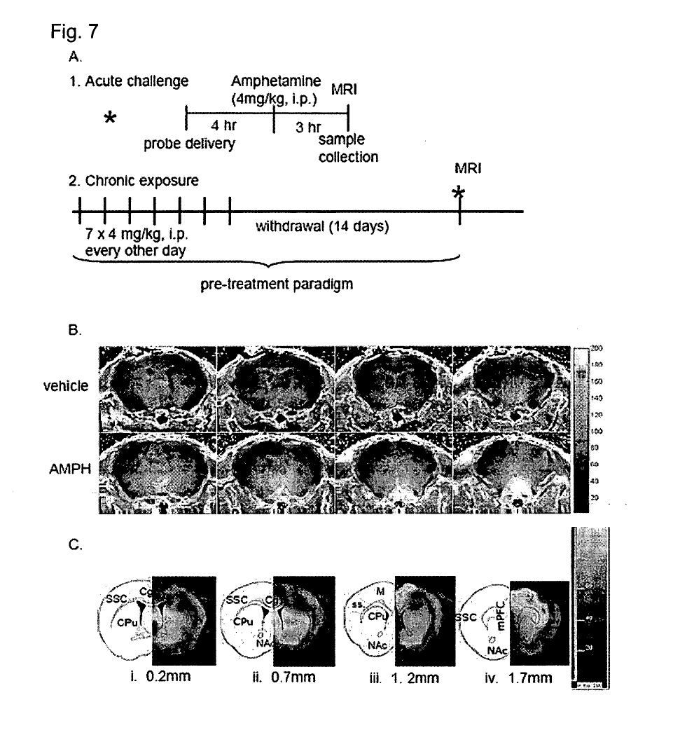

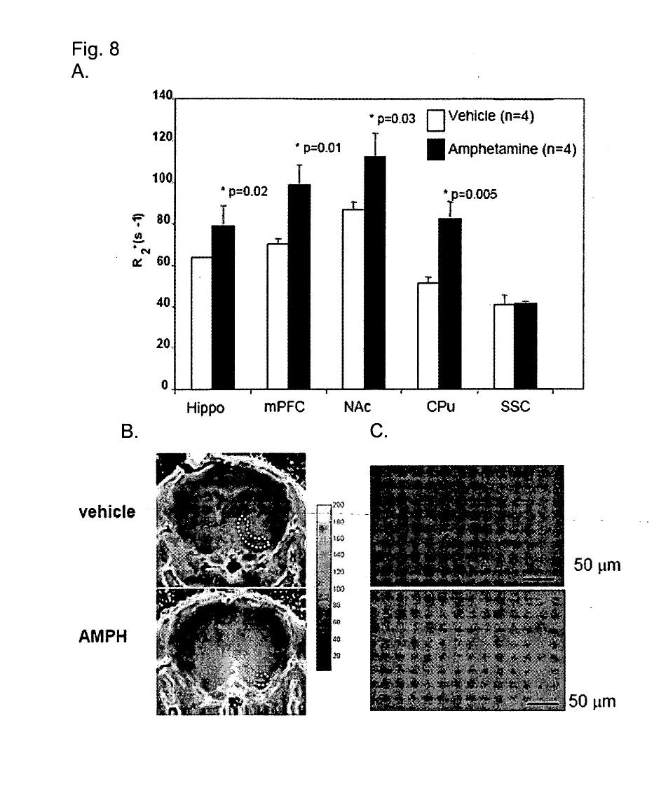

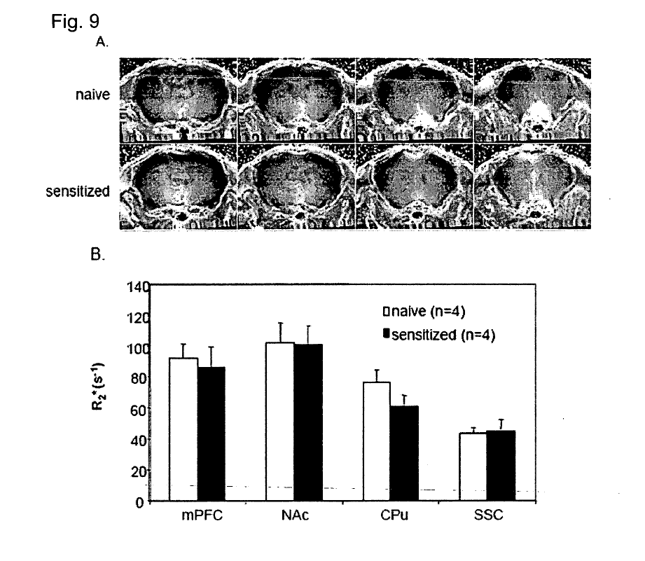

Drug induced gene expression: We studies transcription of fosB and ΔfosB in mice that have no previous exposure (naive) or with prior exposure to amphetamine (sensitized or chronic exposure) (see the Protocol, FIGS. 7A1-7A2). FIG. 7B shows representative R₂* maps in live animals (from the bregma [0 mm] to 1.0 mm anterior) seven hours after the infusion of SPION-fosB in naive animals treated with amphetamine (AMPH) and saline (vehicle). Subtraction maps show various regions in the contralateral hemisphere with elevated retention in naive animals (FIG. 7C, i-iv). Most consistently is the statistical analysis revealing the hippocampus (Hippo), mid prefrontal cortex (mPFC), nucleic accumbence (NAc), caudate putaman (CPu), but not the SSC (FIG. 8A). The elevation of SPION-fosB retention in the naive group after amphetamine exposure is also shown by the immunohistological elevation of fosB mRNA in the shell of the NAc after amphetamine (FIGS. 8B and 8C). FIG. 9A shows an equal elevation of amphetamine-induced SPION-fosB retention in the naive and sensitized (with withdrawal) animals. The elevation presents no significant difference (FIG. 9B).

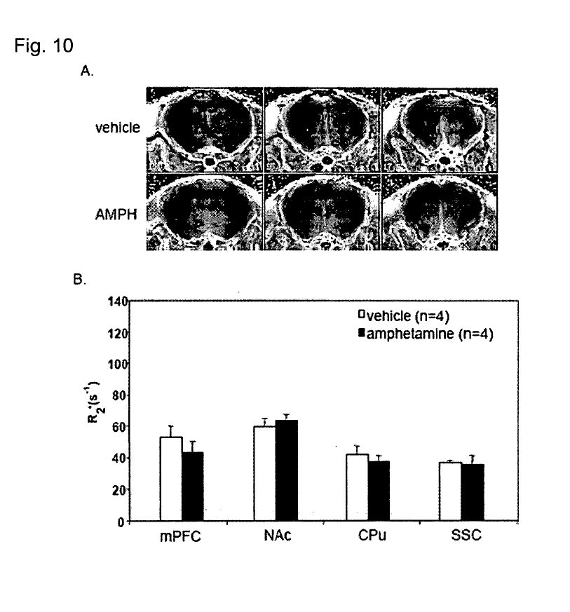

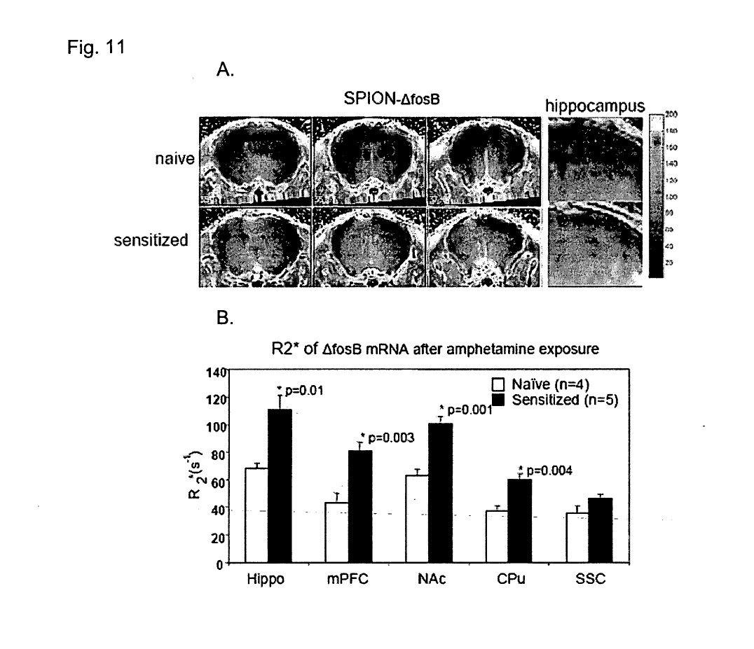

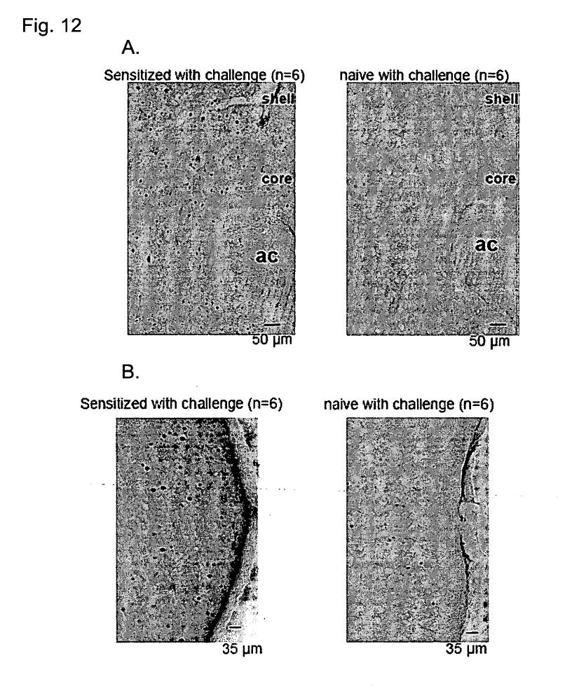

We then compared ΔfosB mRNA transcription in the naive and sensitized animals (FIG. 7A2, repeated administration of saline or amphetamine during chronic exposure, respectively). There was no induction of ΔfosB mRNA after amphetamine exposure in the naive animals (FIGS. 10A & 8B). However, the retention of SPION-ΔfosB is significantly elevated in sensitized (chronic exposure with withdrawal) animals after the drug in all ROI except SSC (FIGS. 11A and 11B). The data shows that amphetamine challenge after sensitization can induce the transcription of ΔfosB mRNA and translation to its protein; two representative region of the pleasure and reward pathway of the brain are shown in FIGS. 12A and 12B.

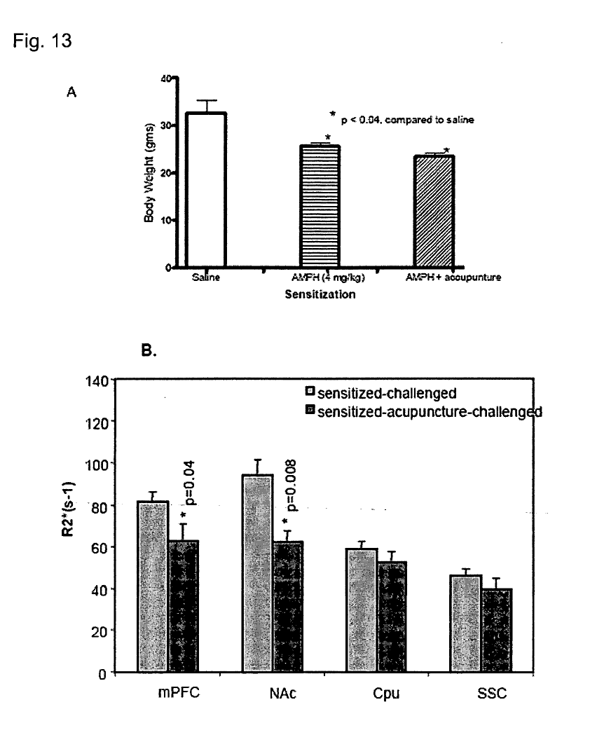

We recorded and analyzed body fluctuations in three groups of animals. FIG. 13 shows amphetamine sensitization significantly causes weight drops by approximately 10 grams compare to the group that received saline. This behavior matches what is observed in chronic amphetamine users. Acupuncture analgesia to mice during their amphetamine withdraw (once every other day) shows ΔfosB mRNA activation is significantly reduced in the mPFC and NAc, but not in the CPu and SSC (FIG. 13B). The reversible of gene transcription elevation by acupuncture was not accompanied by a gain of body weight (FIG. 13A). Acupuncture analgesia was achieved by inserting stainless steel needles (diameter: 0.25 mm) bilaterally to a depth of 5 mm into the animal's hind leg near the knee joint. Constant current with square wave electric stimulation apparatus (HANS, China) were then applied via the two needles for 25 minutes with currency of 10 μm and a frequency of 2 Hz. For each animal, electro-acupuncture may be repeated every other day for a total of seven Electro-acupuncture treatments.

### Example 5

### Neurogenesis: Using SPION-Nestin to Detect Cells Expressing Nestin mRNA

### Introduction:

Stem cells of the brain can differentiate into three major neural lineages: neurons, astrocytes and oligodendrocytes. In most regions of the CNS, neural stem cells almost exclusively give rise to glial cells. These types of cells can be detected using SPION-gfap and SPION-nestin. The generation of new neurons, however, from neural stem cells (neurogenesis) is restricted to two areas of the adult CNS: the subgranular zone of the hippocampus dentate gyms (SGZ; also known as the subgranular layer, SGL) and the subventricular zone (SVZ) of the lateral ventricle. Proliferating and fate determination in stem cells in the SGZ give rise to transit amplifying cells that differentiate into immature neurons, which migrate into the granule cell layer of the dentate gyms where they mature and integrate into new granule neurons.

SVZ is another source of neural stem cells in the process of adult neurogenesis. SVZ has the largest population of stem cells in the adult brain of rodents, monkeys and humans. Four cell types have been described in the SVZ: (1) ciliated ependymal type E cells facing the lumen of the ventricle, whose function is to circulate the cerebrospinal fluid and provide inhibitor of glial differentiation; (2) proliferating type A neuroblasts, expressing PSA-NCAM, Tuj1, and Hu, and migrating in “chains” toward the olfactory bulb (OB); (3) slowly proliferating type B cells expressing nestin and GFAP, and unsheathing migrating type A neuroblasts; and (4) actively proliferating type C cells or “transit amplifying progenitors” expressing nestin, and forming clusters interspaced among chains throughout the SVZ.

Neurons generated in SVZ travel to the olfactory bulb via the rostral migratory stream, which has recently found in humans. Proliferating stem cells in the SVZ, with the aids of inhibitors from the ependymal ciliated cells, give rise to transit amplifying cells (type C) and differentiate into typeA neuroblasts. Type A neuroblasts, in the channel formed by type B cells, migrate through the rostral migratory pathway to the olfactory bulb where they differentiate into local interneurons in the granular layer and the periglomerular layer; to olfactory sensory neurons, tufted neurons, mitral neurons, granule neurons, and periglomerular neurons. Therefore, detecting cells expressing nestin mRNA along with Actin mRNA or GFAP using the method of the present invention can be used to find the region expressing stem cell activity in the brain and to further study the neurogenesis process.

### Methods and Results

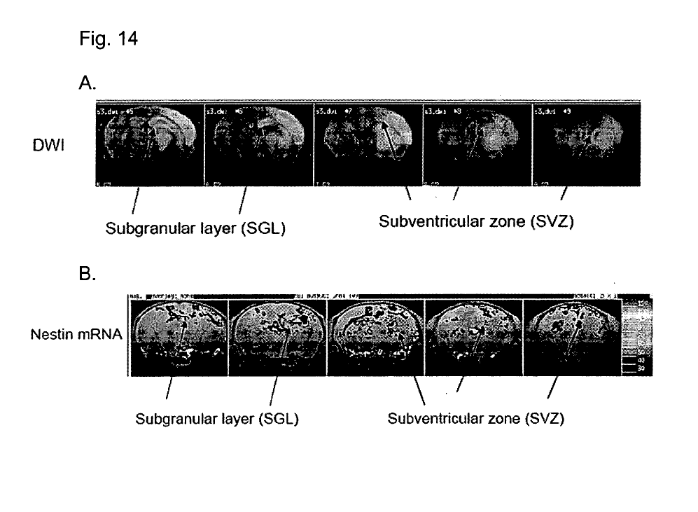

We induced global cerebral ischemia condition simulating cardiac arrest in C57black6 mice for 30 or 60 minutes. We then observed the resulting brain edema in a representative mouse (FIG. 14A). FIG. 14A shows images detected by DWI/MRI at two days after GCI induced by a sixty minute BCAO. This model simulates cerebral injury after cardiac arrest or heart attack, because brain injury in this model can be reversed by hypothermia, a condition that can also reverse brain damage during cardiac arrest. We developed an MR contrast agent targeting nestin expressing cells (T2 susceptibility agent with sODN-nestin, which has a sequence complementary (antisense) to nestin protein (sODN-nestin, 5′-tcccaaggaaatgcagcttctgctt-3′, SEQ ID NO:7) mRNA of the mouse). Four weeks after GCI, we acquire baseline R2* maps, then we delivered SPION-nestin (2 mg Fe per kg) via eye drops (FASEB J 22:1193-1203, 2008). We then acquired additional R2* maps the next day. Subtraction R2* maps show cell expressing nestin mRNA are detected in the SVZ and SGL (FIG. 14B).

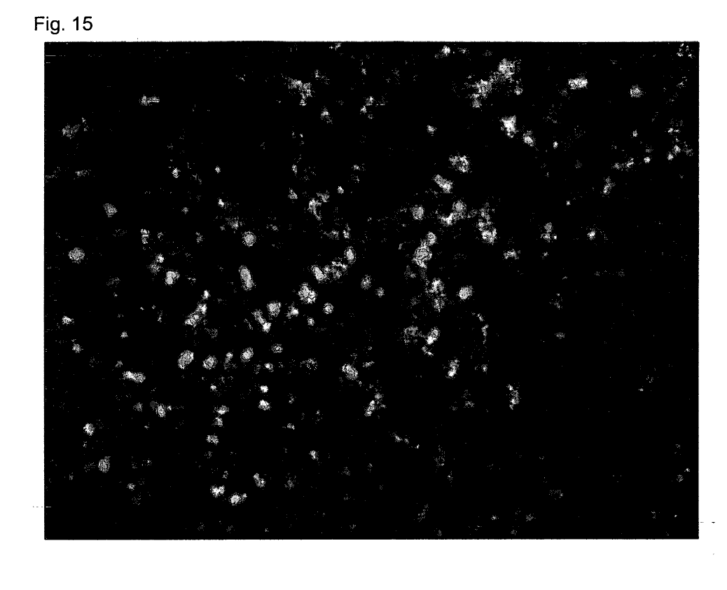

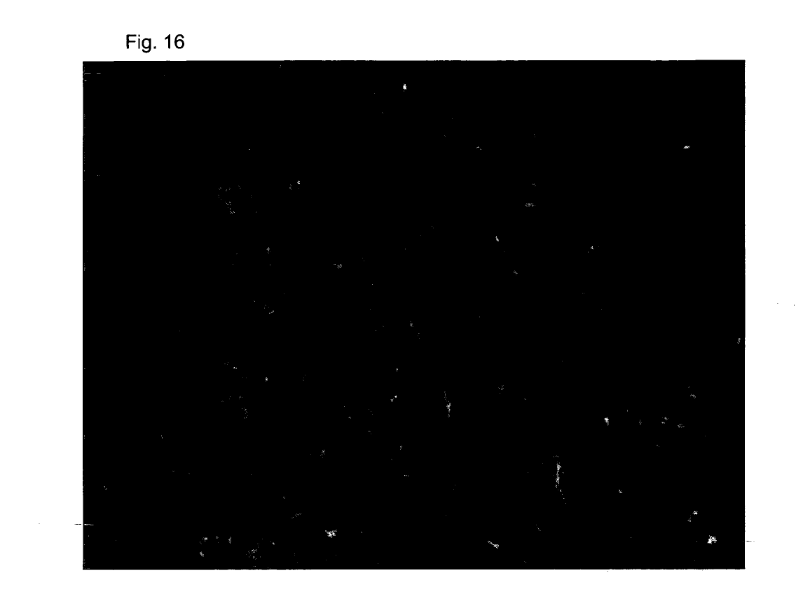

Histological examination of mouse brain SVZ from mice after GCI shows nestin expressing cells binding antibodies against nestin (shown as sharp bright spots in FIG. 15). Nestin antigen does not stain glia cells or nuclei (shown as darker diffuse grey spots in FIG. 15). Therefore, the detected nestin is not produced in gliogenesis. It appears that nestin-producing stem cells are present in the SVZ and SGL of mice that experienced GCI (i.e. 30 min seen in FIG. 15), but nestin producing stem cells are absence in sham-operated mice (FIG. 16, note no sharp bright spots).

Although the invention has been described in connection with specific embodiments, it is understood that the invention is not limited to such specific embodiments but encompasses all such modifications and variations apparent to a skilled artisan that fall within the scope of the appended claims.

1. A method for delivering a targeting nucleic acid to the brain tissue of a human or non-human animal having a leakage in the blood brain barrier wherein the targeting nucleic acid is designed for hybridizing to a target nucleic acid in the brain, the method comprising the step of:
administering an active agent that comprises the targeting nucleic acid to an eye of the human or non-human animal wherein the agent travels to the brain tissue of the human and non-human animal and hybridizes to the target nucleic acid if present in the brain.

2. The method claim 1, wherein the agent is administered as an eye drop.

3. The method of claim 1, wherein the target nucleic acid is an mRNA transcribed from a target gene in a brain cell and the targeting nucleic acid is designed to hybridize to a portion of the mRNA.

4. The method of claim 3, wherein the brain cell is selected from a glioma cell, a glia cell, an astrocyte, a neuron, a vascular cell, and a neural stem cell.

5. The method of claim 3, wherein the mRNA transcribed from a gene selected from gfap, actin, mmp-9, neurofilament-1, c-fos, fosb, delta-fosb and nestin.

6. The method of claim 3, wherein the targeting nucleic acid decreases the expression of the target gene.

7. A method for imaging a targeting nucleic acid or a target nucleic acid in the brain of human or non-human animal having a leakage in the blood brain barrier, the method comprising the steps of:
providing an active agent that comprises a targeting nucleic acid and a contrast agent wherein the targeting nucleic acid and the contrast agent are linked together and the targeting nucleic acid can hybridize to a target nucleic acid;
administering the active agent to an eye of the human or non-human animal in an amount sufficient to provide a detectable image;
allowing sufficient time to pass to allow unhybridized active agent to leave the brain; and
imaging the brain wherein a detectable image of the contrast agent in the brain indicates the presence of the target nucleic acid in the brain.

8. The method claim 7, wherein the active agent is administered as an eye drop.

9. The method of claim 7, wherein the human or non-human animal has a disease selected from glioma, brain injury, Alzheimer's disease, multiple sclerosis (MS), viral infection, Huntington's disease, and Parkinson's disease.

10. The method of claim 7, wherein the target nucleic acid is an mRNA transcribed from a target gene in a brain cell and the targeting nucleic acid is designed to hybridize to a portion of the mRNA.

11. The method of claim 10, wherein the brain cell is selected from a glioma cell, a glia cell, an astrocyte, a neuron, a vascular cell, and a neuron stem cell.

12. The method of claim 10, wherein the mRNA is transcribed from a gene selected from gfap, actin, mmp-9, neurofilament-1, c-fos, fosb, delta-fosb, and nestin.

13. The method of claim 10, wherein the targeting nucleic acid decreases the expression of the target gene.

14. The method of claim 7, wherein the targeting nucleic acid has 10 to 100 nucleotides.

15. The method of claim 14, wherein the targeting nucleic acid has 12 to 60 nucleotides.

16. The method of claim 15, wherein the targeting nucleic acid has 15 to 30 nucleotides.

17. The method of claim 7, wherein the contrast agent is a magnetic resonance imaging contrast agent.

18. The method of claim 17, wherein the contrast agent is a superparamagnetic iron oxide particle whose maximum diameter is between 1 nm and 1,000 nm.

19. The method of claim 18, wherein the particle is selected from a monocrystalline iron oxide nanoparticle, an ultrasmall superparamagnetic iron oxide particle, and a cross-linked iron oxide particle.

20. The method of claim 18, wherein the maximum diameter of the particle is between 10 nm and 100 nm.

21. The method of claim 18, wherein the contrast agent further comprises cross-linking dextran surrounding the particle.

22. A method for decreasing the expression of a target gene in the brain of a human or non-human animal having a leakage in the blood brain barrier, the method comprising the step of:
administering an active agent comprising a targeting nucleic acid to an eye of the human or non-human animal in an amount sufficient to decrease the expression of a target gene wherein the targeting nucleic acid can hybridize to a target nucleic acid corresponding to the target gene in the brain and the hybridization between the targeting nucleic acid and the target nucleic acid leads to decreased expression of the target gene.

23. The method claim 22, wherein the active agent is administered as an eye drop.

24. The method of claim 22, wherein the human or non-human animal has a viral infection or stroke.

25. The method of claim 22, wherein the target nucleic acid is an mRNA transcribed from the target gene in a brain cell and the targeting nucleic acid is designed to hybridize to a portion of the mRNA.

26. The method of claim 25, wherein the mRNA is transcribed from a gene selected from a viral gene or a polyADP-ribose polymerase gene.

27. The method of claim 22, wherein the targeting nucleic acid is selected from an antisense nucleic acid, an si RNA, and an shRNA.

28. The method of claim 22, wherein the targeting nucleic acid has 10 to 100 nucleotides.

29. The method of claim 28, wherein the targeting nucleic acid has 12 to 60 nucleotides.

30. The method of claim 29, wherein the targeting nucleic acid has 15 to 30 nucleotides.

31. The method of claim 22, wherein the active agent further comprises a contrast agent linked to the targeting nucleic acid for tracking the delivery of the targeting nucleic acid.

32. The method of claim 31, wherein the contrast agent is an MRI contrast agent.

33. A method for treating a disease or disorder in the brain of a human or non-human animal having a leakage in the blood brain barrier, the method comprising the step of:
administering an active agent to an eye of the human or non-human animal in an amount sufficient to treat the disease or disorder wherein the active agent comprises a targeting nucleic acid and a therapeutic agent linked together and the targeting nucleic acid can hybridize to a target nucleic acid located at the site of the disease or disorder.

34. The method claim 33, wherein the active agent is administered as an eye drop.

35. The method of claim 33, wherein the human or non-human animal has a disease selected from glioma, brain injury, Alzheimer's disease, multiple sclerosis (MS), viral infection, Huntington's disease, and Parkinson's disease.

36. The method of claim 33, wherein the target nucleic acid is an mRNA transcribed from a target gene in a brain cell and the targeting nucleic acid is designed to hybridize to a portion of the RNA.

37. The method of claim 36, wherein the brain cell is a glioma cell or a cell infected by a virus.

38. The method of claim 36, wherein the mRNA is transcribed from a gene selected from gfap or a viral gene.

39. The method of claim 36, wherein the target nucleic acid decreases the expression of the target gene.

40. The method of claim 33, wherein the target nucleic acid has 10 to 100 nucleotides.

41. The method of claim 40, wherein the target nucleic acid has 12 to 60 nucleotides.

42. The method of claim 41, wherein the target nucleic acid has 15 to 30 nucleotides.

43. The method of claim 33, wherein the therapeutic agent is a cytotoxic agent or a chemotherapy agent.

44. The method of claim 33, wherein the active agent further comprises a contrast agent linked to the targeting nucleic acid, the therapeutic agent, or both for tracking the delivery of the active agent.

45. The method of claim 44, wherein the contrast agent is an MRI contrast agent.

46. A method for delivering a nucleic acid comprising a nucleotide sequence encoding a polypeptide to the brain of a human or non-human animal having a leakage in the blood brain barrier, the method comprising the step of:
administering an active agent comprising the nucleic acid to an eye of the human or non-human animal in an amount sufficient to express the polypeptide in the brain of the human or non-human animal.

47. The method of claim 46, wherein the nucleic acid further comprises a promoter operably linked to the nucleotide sequence.
